(19) United States
(12) Patent Application Publication
(10) Pub. No.: US 2018/0279976 A1
(43) Pub. Date: Oct. 4, 2018

(54) INTRAORAL SENSOR POSITIONING SYSTEM

(71) Applicant: Todd C. Miller, San Jose, CA (US)

(72) Inventor: Todd C. Miller, San Jose, CA (US)

(21) Appl. No.: 15/659,217

(22) Filed: Jul. 25, 2017

Related U.S. Application Data
(60) Provisional application No. 62/462,238, filed on Feb. 22, 2017.

(51) Int. Cl. A61B 6/14; A61B 5/00; A61B 6/00

(52) U.S. Cl. CPC A61B 6/145; A61B 6/40; A61B 6/587; A61B 5/0088

(57) ABSTRACT

A sensor positioner for use with a dental digital radiography system includes an x-ray source which transmits through an x-ray cone an x-ray stream which has a generally circular cross-section and an intraoral sensor which is aligned with the x-ray source. The sensor positioner includes an aiming ring and a sensor carrier. The aiming ring includes a receptacle which is a single hole having a predefined cross-sectional shape with two axes of symmetry wherein the aiming ring is capable to being disposed adjacent to and visually aligned with to the x-ray source through the x-ray cone. The sensor carrier includes an attachment rod which has a first end and a second end and which has the same shape as the receptacle of the aiming ring so that the receptacle receives the attachment rod at the first end in one of two ways. The sensor holder is coupled to the second end of the attachment rod to integrally form the sensor carrier. The sensor holder has a surface on which the intraoral sensor is placed which visually aligns the intraoral sensor and a clip which secures the dental sensor to the surface so that the intraoral sensor does not slide relative to the center of the aiming ring.

### BACKGROUND OF INVENTION

### Field of the Invention

This invention relates to an intraoral sensor positioning system for dental x-ray procedures which assures a fixed relationship between the x-ray equipment and an intraoral sensor and which also assures that an individual sensor holder is properly positioned in the patient's mouth with respect to the x-ray equipment.

### Description of the Prior Art

Dental radiographs are made using x-ray examination units suited for dental purposes. Dental x-ray films have been developed for intraoral shots and have a generally plate-like construction and standardized dimensions so that the film can be introduced into the oral cavity.

In one procedure, the film alone is placed within the patient's mouth and held in place proximate the tooth or teeth to be filmed when the patient closes her mouth over the film. It has been determined that proper orientation of the film with respect to the tooth or teeth to be filmed parallel to the longitudinal axis of the tooth, is required to eliminate distortions, improper focus, and the like. The film carriers are used including a back plate for mounting the x-ray film and a bite plate orientated at a right angle to the back plate. The x-ray film is held against the back plate, the film carrier is introduced at the proper location within the patient's mouth and the patient is instructed to bit down on the bite plate. There is still a need to assure proper orientation of the x-ray tube with respect to the x-ray film mounted against the back plate.

U.S. Pat. No. 4,965,885 teaches a film carrier which engages one end of a guide rod at a different location on the film carrier by means of a plurality of spaced bores in the bite plate. The other end of the connecting rod is slide-ably received through an orifice in a sighting ring affixed circumferentially around an x-ray cone.

U.S. Pat. No. 5,327,477 teaches an x-ray film positioning system that includes a carrier and a sighting ring (aiming ring) that receives the carrier to address misalignment of the receptor with respect to the x-ray source. Annotations are displayed on the sighting ring corresponding to holes in the ring as well as corresponding carriers received in those holes. The film is placed on the carrier and is held between a frictional support member and a film support plate. With the film loaded on the carrier, the carrier is inserted into its corresponding annotated hole in the sighting ring in order to position the film within a projection of a window of the ring. One of ordinary skill in the art will appreciate that the arrangement does not ensure that the film will be centered with respect to the film support plate. There is no assurance that the film will be centered with the window of the sighting ring. If the film is not centered with the film support plate. Even if the carrier is inserted into its corresponding hole in the ring, the film will not be centered with the x-ray source and the x-ray image produced will be off-centered resulting in the need to take further x-rays, which is not desirable. Conventional positioning devices are prone to causing error in a clinical environment. Dental radiographs are made using x-ray examination units including an x-ray cone or tube positioned proximate the patient and aligned to take x-rays of certain teeth. Dental x-ray films developed for intraoral shots generally have a plate like construction and standardized dimensions so that the film can be introduced into the oral cavity. In one procedure, the film alone is placed within the patient's mouth and held in place proximate the tooth or teeth to be filmed when the patient closes her mouth over the film. It has been determined, the proper orientation of the film with respect to the tooth or teeth to be filmed parallel to the longitudinal axis of the tooth, is required to eliminate distortions, improper focus and the like. The film carriers (“bite blocks”) which are used have a back plate for mounting the x-ray film and a bite plate orientated at the right angle to the back plate. The x-ray film is held against the back plate, the film carrier is introduced at the proper location within the patient's mouth, and the patient is instructed to bite down on the bite plate. The film carriers have different shapes depending on the area of the mouth to be filmed, e.g., anterior, vertical bite wing, upper left/lower right or upper right/lower left and bite wing film carriers. There is a need to insure proper orientation of the x-ray tube with respect to the x-ray film mounted against the back-plate. The procedure for accomplishing this goal is not always straight forward. The film positioning system which U.S. Pat. No. 5,327,477 teaches greatly simplified this difficult to understand prior art procedure by engineering the device in which there are three rod holders each of which is annotated to match a specific film holder. The film positioning system for dental x-ray procedures includes an x-ray tube aiming device (aiming ring) and a plurality of film carriers. Each of the film carriers includes a film holder on one end and an attachment rod extending therefrom. One of a plurality of distinct attachment rod holders separately positions each of film carriers in the proper orientation with respect to the x-ray tube.

Referring to FIG. 1 an x-ray film positioning system 6 includes an attachment rod 11 and a film carrier 12 which is configured for the anterior portion of the mouth. A bite block portion 14 is located on the distal end 16 and includes a film holder support plate 18 and a biting surface 20. The attachment rod 11 extends from the bite block 14 and is preconfigured for proper orientation of the bite block 14 with respect to a sighting ring (aiming ring) 22. The sighting ring 22 is circumferentially engageable about an x-ray tube and includes a positioning mechanism 24 for automatically positioning the film carrier 12 in the proper orientation with respect to the line of sight of the x-ray cone shown by a line of sight 26. The positioning mechanism 24 includes film carrier receptacles in the form of rod holders 28, 30 and 32. Each of the rod holders 28, 30 and 32 includes an orifice 34 therethrough. The attachment rod 13 passes slide-ably within one of the rod holders 28, 30 and 32 or even completely through as shown by arrow 34. The film carrier 12 is preconfigured such that when fit into one of the rod holders 28, 30 and 32 the line of sight 26 between the center of the sighting ring 22 and the center of the support plate 18 is such that proper orientation of the film (not shown) with respect to the teeth of interest and proper orientation of the film with respect to the x-ray cone is assured. This configuration helps in assuring all four corners of the x-ray film are exposed. The film carrier 12 is particularly suited to filming the anterior portion of the patient's mouth. The upper anterior may be filmed when the patient bites down on the bite surface 20. In this orientation, the film support plate 18 is proximate the upper teeth. Rotating the aiming ring 180 degrees on the x-ray tube places the film carrier 12 in the proper position for filming the lower anterior teeth.

Referring to FIG. 2 in conjunction with FIG. 1 and FIG. 3 a film carrier 40 is preconfigured for filming either the upper left or lower right posterior portions of the patient's mouth. There is a plurality of film carriers each of which is configured such that when placed in the appropriate rod holders 28, 30 and 32 the proper orientation of the film within the patient's mouth is assured. To guarantee that the x-ray technician correctly places the correct the film holder in the proper orientation with respect to the positioning mechanism 24 of the aiming ring 22 each of the film holders is annotated. The embossed or molded annotation 42 on a bite plate 44 is “UL”, and “LR”, corresponding respectively to “upper right”, and “lower left” filming within the patient's mouth. The “3” corresponds to the rod holder 28 which is also annotated with a “3” as shown at 46. The film holder 12 would have the annotation “2”, “ANT” assuring that the technician uses the film holder 12 for anterior shots and that the film holder 12 is properly placed in the rod holder 30 which has a complementary annotation “2”. The technician does not have to adjust the film holder with respect to the sighting ring 22 for proper orientation and much of the guess work of dental radiography is eliminated. If the dentist orders lower right posterior x-rays of the patient, the technician selects film carrier 40 and notes the “LR” corresponding to lower right with the film in place against the film support surface plate 48. A bite block 50 is placed proximate the desired location on the right side of the patient's mouth. When the patient bites down on the biting surface 52, the film support plate 48 is correctly and automatically positioned proximate the patient's lower teeth on the right side. Noting the annotation “3” the technician places the attachment rod 46 in the rod holder 28 which is annotated with a “3”. The aiming ring 22 is then slid onto the x-ray cone. In this way, a more stable system is achieved insuring proper X-ray film alignment and system accuracy. Time is saved and patient comfort is increased because the numerous adjustments required in prior art systems are not required in the single ring film positioning system. There is a fixed relationship between the x-ray film and the portion of the patient's mouth to be filmed and a precise orientation between the x-ray equipment and the x-ray film. A single sighting ring 22 accepts several uniquely configured film holders thereby insuring the proper position of each film holder. The guess work of determining the proper orientation is eliminated by the distinct film holder receptacles on the sighting ring 22 each of which is annotated for association with its corresponding film holder. Film movement is reduced and retakes resulting in unnecessary radiation exposure are not required.

Referring to FIG. 4 a film carrier 60 generally includes a film holder or a bit block portion 62 having a film support plate 64 extending perpendicularly from a biting surface 66. An attachment rod 68 extends in a preconfigured pattern from the bite block portion 62. A frictional support member 70 is located at the intersecting gap 72 between the biting plate 66 and an offset film support plate 64 and releasably secures a film 74 against the film support plate 64 by means of a frictional fit within the intersecting gap 72. This configuration allows injection molding or similar manufacturing processes of the film carrier 60 resulting in a preferred and cost efficient unitary configuration. The frictional support member 70 is biased for releasably securing the film 74 against support plate 64.

Referring to FIG. 5 this x-ray film positioning system 6 also includes a sleeve 80 which encompasses at least a portion of a bite block 82, namely a support plate 84, thereby assisting in keeping a film 86 in the proper orientation on the support plate 84. The sleeve 80 includes an opening 90 which receives the film support plate 84 and the film 86. The opening 90 leads to an inner cavity 81 which has width and depth dimensions approximately the same as those of the x-ray film 86 so that x-ray film 86 fits snugly therein to hold the x-ray film 86 in place and protect the patient from the generally sharp film edges. The sleeve 80 assists in greater x-ray fidelity by holding the x-ray film 86 more firmly against the support plate 84. The sleeve 80 insures a more sanitary system since it can be economically disposed of after use on one patient. The sleeve 80 prevents the x-ray film 86 from becoming wetted with saliva facilitating the film development process. The sleeve 80 holds the x-ray film 86 firmly and comfortably in place by encompassing the exposed edges of the x-ray film 86 with a soft, pliable sleeve having an opening approximately the size of the x-ray film 86 so the x-ray film 86 can slide into the sleeve 80. The sleeve has rounded edges 83 and 85 which increase patient comfort. The sleeve 80 may be flavored. A flavoring agent could be placed proximate the sleeve 80 during packaging so that the favoring agent could leach onto the sleeve 80. Alternatively, a flavoring agent could be added during manufacture of the sleeve 80 for masking an otherwise plastic or rubber taste.

Referring to FIG. 6 in conjunction with FIG. 7 an aiming ring 94 includes a unique beveled interior surface 96 defining a radius R1 at the rear of aiming ring 94 which faces the x-ray tube and a smaller radius R2 at the front of the aiming ring 94 which faces the patient. A ridge 98 in conjunction with the beveled surface 96 insures a frictional fit of the aiming ring 94 on x-ray tube 60 which generally has a complementary configuration, namely a sloping surface 62 having the smallest diameter at its end 64 which is placed proximate the patient's mouth. The aiming ring 94 has a circular shape which receives a tube-shaped x-ray head 60. The aiming ring 94 could also be square shaped or otherwise properly configured for temporary attachment to other known x-ray devices. Other film carrier positioning mechanisms are affixable to the x-ray device including a plurality of distinct attachment rod holders. The aiming ring 94 may be configured to circumferentially engage most common x-ray tubes and other adapters may be designed to affix the aiming ring 94 to x-ray tubes of other configurations.

Referring to FIG. 8 in conjunction with FIG. 1, FIG. 9, FIG. 6, FIG. 11 and FIG. 12 to assure accurate and defined positioning of the x-ray film within the patient's mouth, the plurality of correctly positioned rod holders 28, 30 and 32 allow the use of distinctly configured film carriers such as an anterior film carrier 110, a posterior upper left and lower right film carrier 112, an upper right and lower left film carrier 114 and a bite-wing film carrier 116. Vertical bite-wing holders may also be included. The anterior film carrier 110 is correctly mated with the rod holder 30, (position 2) of the aiming ring 22 which is positioned on the x-ray tube such that the rod holder 30 is proximate the 6 o'clock position for filming the upper anterior teeth. For filming the lower anterior teeth, rod holder 30 is positioned proximate the 12 o'clock position. The posterior upper left and lower right film carrier 112 is mated with the rod holder 28 (position 3) of the aiming ring 22 which is positioned on the x-ray tube such that the rod holder 28 is proximate the 9 o'clock position for filming the lower right teeth, and positioned proximate the 3 o'clock position for filming the upper left teeth. The posterior lower left and upper right film carrier 114 is mated with the rod holder 32 (position 1) of the aiming ring 22 which is positioned on the x-ray tube such that the rod holder 32 is proximate the 9 o'clock position for filming the upper right teeth and positioned in the 3 o'clock for filming the lower left teeth.

Referring to FIG. 13 in conjunction with FIG. 1 although the positioning mechanism are related to rod holders 122, 124 and 126. Each of the rod holders 122, 124 and 126 has an orifice 128 which allows for proper longitudinal positioning of a film plate 130 with respect to x-ray tube 132. Proper dimensioning of the aiming ring 22 with respect to an x-ray tube 132 would automatically fix the proper distance between the x-ray tube lens and the film support plate 130 further reducing the need for longitudinal adjustment. The film carrier rod holder 124 has a cavity 125 which does not extend completely through the aiming ring 127. Instead of annotations each attachment rod could be uniquely configured for mating only with its correct corresponding rod holder. This could be accomplished by using different shaped receptacles in the aiming ring corresponding with each rod.

Referring to FIG. 14 in conjunction with FIG. 12 the method of utilizing the system includes the step 150 of selecting the proper film carrier for the set of teeth to be filmed, the step 152 of placing the film in its proper location of the bite block and the protective sleeve the film/bite block combination, the step 154 of engaging the attachment rod into the corresponding rod holder of the aiming ring, the step 156 of placing the bite block within the patient's mouth, the step 158 of guiding the x-ray tube to the aiming ring and the step 160 of exposing the film. In this way, the method assures first that the film does not move with respect to the film holder once placed in the patient's mouth and that the x-ray tube is maintained in the proper position with respect to the patient's mouth and the film carrier for accuracy. The system assures a fixed relationship between the x-ray tube and the film holder limiting radiation exposure, guaranteeing accuracy, and eliminating patient discomfort. The aiming ring 22 and the film holders 40, 110, 112, 114 and 116 may be made of the same thermoplastic or thermosetting materials or similar including poly-propylene, manufactured inexpensively by known extrusion, injection or autoclave methods and sold either as kits or separately. The film holders could be considered disposable for reduced disinfection costs and disease transmission. The sleeve 80 may be vinyl or any soft pliable material, such as thermoplastic rubber, for increased patient comfort since sharp edges of the film do not contact sensitive tissue. One material that has been successfully used for the sleeve 80 and that possesses the softness and pliability desired so that the sleeve can stretch over the film and bite block combination is “Kraton” made by the Shell Chemical Company. This sleeve 80 is also disposable n for increased sanitation.

Rinncorp manufactures a XCP-DS digital sensor holder which is positioning device that is configured specifically for a sensor receptor. Each XCP-DS digital sensor holder consists of a sensor grip which includes a bite block upon which a patient can bite down to hold the grip in place in the mouth, a metal arm which has a generally square shaped cross section and an attachment which has a complimentary square shaped opening to receive the square metal arm. The three pieces of the digital sensor holder are color coded to match together as a group. Each color corresponds to a specific radiographic view, such as bitewing, anterior and posterior. A practitioner, such as a dental hygienist, seeking to assemble the digital sensor holder for bitewing x-rays will assemble the three color coded pieces together corresponding to bitewing x-rays. One deficiency of arrangements like the aforementioned-model XCP-DS digital sensor holder is that the square shaped opening in the alignment ring that receives the arm permits the practitioner to easily misalign the grip with respect to the attachment since the square opening will receive the arm in any of four, equally spaced, rotational positions. Because all arms of the model XCP-DS digital sensor holder share the same square cross section, a practitioner can physically couple parts of one color, corresponding to one radiographic view with those of another color, corresponding to a different radiographic view, which may result in the sensor being misaligned with the x-ray source. For obtaining different x-ray images multiple three-piece combinations of grip-arm-attachment must be autoclaved, stored and assembled for use by the practitioner. Often multiple x-rays are taken during a dental examination. The practitioner must be able to carefully assemble the three-piece combination repetitively while ensuring that the parts are properly aligned and not mismatched for the desired radiographic views.

U.S. Pat. No. 9,314,215 teaches a dental positioning system which includes at least one receptor holder having an arm extending from a receptor grip. The receptor grip retains a receptor in a fixed position within the grip and with respect to the arm. The dental positioning system also includes an alignment ring having a window formed therein and having at least one opening corresponding to the at least one receptor holder. The opening is constructed to receive the arm of the receptor holder such that the receptor is substantially centered with respect to the window.

Intra-oral radiographs, i.e., “x-ray images”, are an important aspect of dental care. These x-ray images have many uses, including diagnosing cavities and lesions, documenting treatment, and assisting in guiding procedures, among other things. The x-ray images may be taken using a receptor to receive x-ray radiation emitted from an x-ray radiation source. Such a receptor may include either x-ray sensitive film or an x-ray sensitive electronic image sensor, such as a digital image sensor or intraoral sensor. In the specific case of digital x-ray imaging, a solid-state image sensor (such as an image sensor including a charge-coupled device [CCD] or a CMOS active pixel sensor array [APS]) is used as the receptor instead of x-ray film. Such sensors are typically 5-6 mm thick and have a cable for communication to a processing unit, such as a computer. Electronic sensors, unlike pieces of film, are re-usable and are usually covered by a cross-infection barrier, such as a sheath. Dental intra-oral image receptors are made in several standard sizes. The receptor is positioned in the mouth behind teeth to be positioned incident to x-ray radiation emitted from the x-ray radiation source.

In many applications, it is desirable for the receptor to be positioned substantially perpendicular to the x-ray radiation. If the receptor is not placed and aligned correctly in the patient's mouth, clinically usable x-ray images may not be obtained resulting in having to take additional x-rays with a corrected receptor alignment. Repeated x-ray images are undesirable because of the additional time, labor, material (in the case of film receptors) and increased exposure of the patient's mouth to radiation.

Although prior art techniques and devices may have the capability to achieve usable x-ray images, there are several problems with existing dental x-ray positioning systems, with respect to both film and electronic receptors, which are not addressed adequately by these techniques. Because accurate positioning of the receptor is difficult, several devices have been proposed to assist dental practitioners, such as dentists and hygienists, in placing and aligning the receptor. At least one typical dental x-ray positioning system includes three separable pieces: a grip to hold the receptor having typically a bite block area, a metal arm extending from the grip and an alignment ring being attached to the metal arm from an end of the arm opposite the grip. When positioned in the mouth of a patient, the arm and the alignment ring generally protrude from the mouth and give the practitioner an alignment point on the alignment ring so that the practitioner can position the x-ray radiation source with respect to the positioning system.

Referring to FIG. 15 in conjunction with FIG. 16, FIG. 17 and FIG. 18 U.S. Pat. No. 9,314,215 also teaches a dental positioning system (radiographic positioning system) 200 which includes an alignment ring 202 and a receptor holder 204. The receptor holder 204 is received by the alignment ring 202 while the receptor holder 204 is spaced from the alignment ring 202. The alignment ring 202 has a generally planar form with a plurality of openings 206, each for receiving a receptor holder 204 therein, such as a holder 204. The alignment ring 202 also has a window 208 beside the plurality of openings 206. The window 208 has a generally square or rectangular shape which is surrounded by a circular or curved ridged rim 210 which is used to align with a corresponding shaped portion of a radiation source. The alignment ring 202 includes a frame 209 interposed between the rim 210 and the window 208. The frame 209 centers window 208 within the rim 210. In one embodiment, the frame 209 is constructed as a radiation shield which can direct radiation from the radiation source substantially through the window 208 of the alignment ring 202 during receptor 222 exposure. Some radiation sources have collimators to also filter and direct the radiation from the radiation source toward a receptor. A digital sensor can be used as a receptor. Schick Technologies Inc. manufactures sensors having square or rectangular shapes, such as the CDR Elite digital sensor. The shape of the collimator is often matched to the shape of the receptor so that the collimator will have a square or rectangular shape similar to the shape of the receptor. The polygonal window 208 can be aligned with a collimator having a matching polygonal shape on the radiation source. The alignment ring 202 has a notch 190 formed along an outer edge adjacent to an opening 206. The notch 190 is constructed to receive a portion of a communications device, such as a wireless transmitter, that can communicate with a digital sensor arranged as the receptor 222. The receptor holder 204 positions the receptor for taking anterior x-ray images when the receptor holder 204 is assembled with the alignment ring 202. The receptor holder 204 has an arm 212 with a grip 214 at an end 216 of the arm. The arm 212 of the receptor holder 204 is constructed to be received in an opening 206a of the alignment ring 202 corresponding to the receptor holder 204. The arm 212 of the receptor holder 204 extends from a free end 218 in a direction toward the grip 214. The receptor holder 204 is constructed for sliding movement in opening 206a, and may be further constructed along with the opening 206a so as to be retained in opening 206a by a friction fit. The free end 218 of the arm 212 can be aligned with opening 216a and inserted into and through opening 216a. The arm 212 is suitably formed and dimensioned to dispose the grip 214 at a position with respect to the window 208 such that when the receptor 222 is seated in the grip 214 and the arm 212 is inserted into opening 206a, the center 224 of the window 208 will be aligned with the center 226 of the receptor 222. The arm 212 has a first portion 212a extending from the free end 218, a second portion 212b extending from the first portion 212a, and a third portion 212c extending from the second portion 212b to the grip 214 at end 216. The first and third portions, 212a and 212c, respectively, extend generally perpendicular to the generally planar surface of the alignment ring 202 and the second portion 212b extends generally parallel to the surface of the alignment ring 202 and perpendicular to the first and third portions, 212a and 212c, of the arm 212. The first, second, and third portions lie in a vertical plane that passes through the center of window 208. The receptor holder 204 can be inserted into the alignment ring 202 through the upper keyed opening 206a as shown in FIG. 10. The cross section of the first portion 212a of the receptor holder 204 has a profile shape matching that of opening 216a of the alignment ring 202. There may be a one-to-one correspondence between the receptor holder 204 and opening 206a in the alignment ring 202. By virtue of matching asymmetrical shapes of the opening 206a and the arm 212, the arm 212 is keyed so that it can be inserted only one way in the opening 206a. This keyed arrangement further reduces positioning errors when the alignment ring 202 and receptor holder 204 are coupled together. The opening 206a may also be arranged to restrict entry of the arm 212 of holder 204 from only one side of the opening 206a. Such restriction may further reduce the opportunity for a user to incorrectly couple the receptor holder 204 and alignment ring 202 together. Extending from the third portion 212c of the arm 212, along upper and lower (not shown) surfaces, is a grooved bite block 220 which receives pressure from a patient's teeth to retain the bite block 220. When the receptor holder 204 is inserted into a patient's mouth, the patient bites down on the surfaces of the bite block 220 to retain the receptor holder 204 in position. The sensor grip 214 extends from the third portion 212c of the arm 212, and is arranged to grip a planar receptor 222 such that the receptor 222 extends in a plane parallel with the plane of the alignment ring 202, and substantially perpendicular to a direction of radiation that would be emitted from the radiation source aligned at the rim 210 of the alignment ring 202. The grip 214 described hereinbelow is generally termed an “edge-grip”. The grip 214 has a support base 228 from which extends a pair of fingers 230 and a tab 232, shown most clearly in FIG. 16. The fingers 230 are constructed to be resilient to permit the edges of the receptor 222 to be held firmly in position between the fingers 230. The fingers 230 apply pressure to the edges of the receptor 222 to retain the receptor 222 in a fixed position in the grip 214. It will be appreciated that the grip 214 can be sized to accommodate receptors 222 of different sizes, such as sizes 0, 1, and 2, with a sheath (not shown for clarity of illustration) covering the receptor 222. Where the receptor 222 is a digital sensor, the grip 214 can accommodate sensors such as the aforementioned CDR Elite Sensor which have a sensor active area that has sizes corresponding to the active area of one of size 0, 1, and 2 film receptors. The tab 232 contacts the receptor 222 at a third edge thereof when the receptor 222 is fully seated in the grip 214. While the receptor 222 is positioned between the fingers 230 of the grip 214, the receptor 222 can be translated into engagement with the tab 232 so that the receptor 222 is fully seated in the grip 214. Thus, the tab 232 acts as a physical stop or interference member which provides confirmation to a user that the receptor 222 is fully seated in the grip 214. When fully seated in the grip 214, and when the receptor holder 204 is received in its corresponding opening 206a in the alignment ring 202, the center 226 of the receptor 222 is aligned with the center 224 of the window 208. The following describes one way in which a user might use the system 200. In one embodiment, a user desiring to obtain anterior x-ray images can select receptor holder 204 and can insert receptor 222 in the grip 214 between its fingers 230. The user can then translate the receptor 222 in the grip 214 towards the tab 232 so that the receptor 222 engages the tab 232. When the receptor 222 is gripped between the fingers 230 and engages the tab 232 the receptor is determined to be seated in the grip 214. The user can then insert the free end 218 of the arm 212 of the receptor holder 204 into opening 206a in the alignment ring 202 to couple the receptor holder 204 and the alignment ring 202 together. The coupled system 200 can then be at least partly inserted into a patient's mouth by placing end 216 in the mouth first and positioning the bite block 220 between the patient's teeth at the suitable anatomical location to obtain anterior x-ray images. When the patient bites down on the bite block 220 the user may adjust the distance between the grip 214 and the alignment ring 202 as necessary before aligning the radiation source with rim 210 and window 208. With the system 200 so disposed, the center 226 of the receptor 222 is aligned with the center of the window 224.

U.S. Pat. No. 5,652,779 teaches a film positioning system for dental x-ray and digital radiography procedures which includes several film or digital sensor carriers (bite wing, anterior) each including a film or sensor holder on one end and an extending attachment rod, and an x-ray tube aiming device including an attachment rod holder, the attachment rod and the attachment rod holder keyed to correctly align the film holder with respect to an x-ray cone or tube. One film positioning system includes a non-equilateral pentagon shaped rod holder receptacle and a non-equilateral pentagon shaped attachment rod.

Referring to FIG. 19 in conjunction with FIG. 20, FIG. 21 and FIG. 22 U.S. Pat. No. 5,652,779 also teaches a film positioning system 310 which includes an x-ray tube aiming device 312 and a plurality of film carriers, such as film carrier 314 for vertical bite wing. Other film carries are for upper left/lower right or upper right/lower left, anterior and bite wing. The film carrier 314 includes a film holder 316 and an attachment rod 318 extending therefrom. The attachment rod 318 is received within an attachment rod holder 320 of an aiming device 312. The attachment rod has a non-equilateral pentagon “house” shaped cross section 322 and the attachment rod holder includes a similar non-equilateral pentagon shaped orifice thereby eliminating any uncertainty associated with correctly aligning film holder 316 in a patient's mouth with respect to an x-ray tube placed up against face 313 of aiming device 312. The aiming device 312 is flat plastic plate which could be transparent. The face 313 of the aiming device 312 has cross-hairs 324 and 328 etched therein as well as scored circular grooves 330 and 332 for receiving and/or aligning different diameter circular x-ray tubes. Some x-ray tubes have a square cross section. A groove 334 is used with these types of x-ray tubes. The grooves 334 could instead be printed lines. Additional aiming indicia could be included to accommodate other types of x-ray tubes. In use, the x-ray technician simply places the x-ray film against the film holder 316, places the film carrier 314 within the patient's mouth, attaches the aiming device 312 to film carrier 314 and then brings the aiming tube of the x-ray equipment to bear upon the aiming device 312. There is no guess work associated in trying to determine how the aiming device 312 is to be positioned on the x-ray tube to assure that the film carrier 314 is correctly positioned within the patient's mouth. The upper left/lower right or upper right/lower left film carrier 314a is automatically positioned with respect to the aiming device 312 when the attachment rod holder 320 is in the six o'clock position. The same is true for anterior film carrier 314b. The bite wing film carrier 314c and the vertical bite wing film carrier 314 are correctly positioned when the attachment rod holder 320 is in the nine o'clock position in that the attachment rods are keyed so they only fit in the attachment rod holder one way. Although the attachment rod holder 320 is configured to have a non-equilateral pentagon shaped opening to receive a non-equilateral pentagon shaped attachment rod, other geometric and non-geometric shapes are possible so long as the attachment rod is keyed to fit in the attachment rod holder only one way.

Referring to FIG. 23 an intraoral sensor 360 is mounted on a carrier 362. A wire 364 of the sensor passes through the non-equilateral pentagon shaped hollow rod 318e for connection to a computer used in digital radiography procedures.

U.S. Pat. No. 5,677,537 teaches a device for recording an image in the oral cavity which includes an intraoral sensor with a housing having two main surfaces. The intraoral sensor includes an image recording layer positioned in the housing parallel and adjacent to one of the two main surfaces. A signal line is connected to the housing at one of the two main surfaces remote from the image recording layer. The signal line transmits signals of the image recording layer to a separate image processing and memory unit. A holder with a bite section and a receiving channel in which the intraoral sensor is received is provided. The holder has an abutment surface extending parallel to the two main surfaces. The abutment surface has guide means for aligning the holder and the sensor relative to one another. A hose is slipped over abutment surface and sensor for fastening the sensor at the holder. Such devices are known under the technical “intra-oral sensor”. They are used by dentists to produce x-ray images of individual teeth or groups of teeth.

For this purpose, the housing of the intraoral sensor is inserted into the patient's mouth and positioned behind the tooth or tooth group to be x-rayed whereby the imaging sensor which is embodied planar within the housing faces with its x-ray sensitive layer the tooth or the tooth group. Subsequently, irradiation with x-rays in a minimal dosage is performed from the exterior. The optical impulses which are received by the imaging sensor are transmitted via a signal line extending from the housing to an external image processing and memory unit. This is, in general, a personal computer. In this manner x-ray images can be produced directly on location without any further delay and are immediately visible on the computer monitor. The dentist can then decide if any further images need to be recorded. The various procedures in a dental practice are often very hectic and do not allow the user enough time to deal with technically complicated devices or solutions. In this context, it is often more advantageous to provide a simple solution or design whose functioning is immediately obvious to the user.

Referring to FIG. 24 an intraoral sensor 501 includes a sensor housing 502 with basic dimensions of approximately 28 mm by 39 mm by 7.5 mm. The sensor housing 502 includes two main surfaces 503 and 504 and two long narrow sides and two short narrow sides. In a vicinity of one of the main surfaces 503 an image recording layer that is sensitive to x-rays is arranged within the sensor housing 502. Via a signal line 506 in the form of a flexible cable the signals of the image recording layer 505 can be guided into a non-represented image processing and memory unit. An x-ray source acts in the direction toward the image recording layer 505. The sensor 501 is used such that it is inserted into the oral cavity of a patient with the image recording layer 505 facing the teeth to be x-rayed so that the portion of the jaw to be x-rayed is positioned between the x-ray source and the image recording layer 505. The exact positioning of the axis of the x-ray source on the one hand and of the image recording layer 505 on the other hand can be performed with suitable centering devices.

Referring to FIG. 25 in conjunction with FIG. 24 the device for recording an image in the oral cavity which U.S. Pat. No. 5,677,537 teaches is a centering device is connected to the sensor holder 507 which for this purpose is provided with a plurality of transverse bores 508 into which the centering device can be laterally inserted. The sensor holder 507 is of a one-part construction and includes a bite section 509, an abutment member with abutment surface 510 extending at a right angle to the bite section 509, and a clamping section 511. The clamping section 511 together with the oppositely arranged abutment surface 510 provides a receiving channel 512 into which the sensor 501 can be inserted. Upon insertion of the sensor 501 a preliminary alignment of the sensor 501 and the holder 507 occurs. This is achieved, on the one hand, due to the elasticity of the elastic clamping section 511 and, on the other hand, especially due to the design of the abutment surface 510. The abutment surface 510 is substantially flat and planar and has a size such that the inserted sensor 1 with its main surface 503 rests at the abutment surface 510 which is radiolucent for x-rays. For a lateral alignment of the sensor housing 502 relative to the holder 507 the abutment surface 510 is provided with an edge 513 projecting in the direction toward the sensor 501. The shape of the edge 513 matches the contour of the sensor housing 502 so that when the sensor 501 is inserted the abutment surface 510 with the edge 13 partly encloses the sensor housing 502 to center it.

Referring to FIG. 26 in conjunction with FIG. 24 and FIG. 25 the edge 513 does not extend about the entire periphery of the abutment surface 510, i.e., the lower end does not have an edge portion. Centering of the sensor 501 in the downward direction is achieved by abutment of the corresponding narrow side of the sensor housing 502 at the receiving channel 512. The holder 507 serves for receiving a sensor 501 of an upright design.

Referring to FIG. 27 in conjunction with FIG. 24 and FIG. 25 the holder 507 serves for receiving a sensor 501 for producing images in a landscape format.

Referring to FIG. 28 the edge 513 of the abutment surface 510 serves as a preliminary fixation means of sensor 501 and holder 507 relative to one another. The final fastening is achieved with a hose 514 made of natural rubber which is slipped first over the abutment surface 510 and the sensor housing 502 and which is pulled preferably over the bite section 509. The own elasticity of the hose 514 is sufficient for pulling the sensor 501 securely against the abutment surface 510 so that the holder 507 and sensor 501 are secured in a defined alignment relative to one another. The signal line 506 which is guided away from the sensor housing 502 from the second main surface 504 is guided through a channel 515 which is provided at the underside of the bite section 509. The signal line 506 is clamped between the channel walls 516.

Referring to FIG. 29 in the holder 507 the abutment surface 510 and the bite section 509 are not positioned at a right angle to one another, but the bite section 509 is positioned at a slant to the abutment surface 10 for the sensor 501. This holder 507 is designed for use in the molar area and especially suitable for image recording of molars.

U.S. Pat. No. 8,016,483 a dental bite block and sensor holder assembly which has a bite block configured to detachably affix to a sensor holder. Affixing surfaces are provided between the bite block and the sensor holder such that the sensor holder is removably affixed to the bite block. An alternative guide arm has more than one attachment site for removably affixing a sensor holder to the guide arm.

U.S. Pat. No. 9,354,506 teaches a system for holding and aligning a radiation sensing device which includes a radiation sensing device having a sensor engagement member and a holder. The holder has a retention member including first and second retention guides connected with opposing ends of a back plate. The first retention guide also includes a complementary holder engagement member configured to mate with the sensor engagement member at a preset position.

U.S. Pat. No. 4,965,885 teaches a film carrier capable of engaging with one end of a guide rod in a different location on the film carrier by means of a plurality of spaced bores in the bite plate. The other end of the connecting rod is slide-ably received through an orifice in a sighting ring affixed circumferentially around the X-ray cone. The U.S. Pat. No. 3,745,344 and U.S. Pat. No. 1,947,014 teach similar constructions.

One problem with such constructions is that the x-ray technician is still left with the task of assuring proper a orientation of the film carrier within the patient's mouth and also a proper orientation between the film carrier and the aiming ring. The x-ray technician, although skilled, may not always be able to assure proper orientation of the film carrier in the patient's mouth or proper orientation of the film carrier with respect to the aiming ring as it presently has been constructed in prior art devices.

In some prior art devices, two positioning procedures must be performed. First, the guide rod must be placed in the proper bore in the biting plate. Second, the guide rod must be properly positioned within the aiming ring to assure the correct distance between the x-ray cone and the film. If the doctor orders x-rays of the upper right, lower left and anterior portions of the patient's mouth, it may not always be apparent to the technician which arrangements and configurations are proper for best filming. Worst, once the proper adjustments are made, realignment may be needed if the patient moves his head or the guide rod improperly moves with respect to the aiming ring. Improper film orientation resulting from the use of such devices is inefficient, results in unnecessary radiation exposure, and patient discomfort. The technician may not be aware of movement resulting in improper exposures, and the need for additional X-rays may not be known until the x-ray plates are exposed. Another related problem is that prior art devices may require a separate uniquely configured aiming ring for each film holder. This adds to the overall expense of prior art systems and increases the possibility of confusion in matching the correct film holder with the correct aiming ring. Intra-oral radiographs, i.e., “x-ray images”, are an important aspect of dental care. Such x-ray images have many uses, including diagnosing cavities and lesions, documenting treatment, and assisting in guiding procedures, among other things. The x-ray images may be taken using a receptor to receive x-ray radiation emitted from an x-ray radiation source.

In many applications, it is desirable for the receptor to be positioned substantially perpendicular to the x-ray radiation. If the receptor is not placed and aligned correctly in the patient's mouth, clinically usable x-ray images may not be obtained, which may result in having to take additional x-rays with a corrected receptor alignment. Repeated x-ray images are undesirable because of the additional time, labor, material (in the case of film receptors) and increased exposure of the patient's mouth to radiation.

Although prior art techniques and devices may have the capability to achieve usable x-ray images, there are several problems with existing dental x-ray positioning systems, with respect to both film and electronic receptors, which are not addressed adequately by these techniques. Because accurate positioning of the receptor is difficult, several devices have been proposed to assist dental practitioners, such as dentists and hygienists, in placing and aligning n the receptor. At least one dental x-ray positioning system includes three separable pieces which are a grip to hold the receptor, a metal arm and an alignment ring. The receptor may have a bite block. The metal arm extends from the grip. The alignment ring attaches to the metal arm from an end of the arm opposite the grip. When positioned in the mouth of a patient, the metal arm and the alignment ring generally protrude from the mouth and give the practitioner an alignment point on the alignment ring so that the practitioner can position the x-ray radiation source with respect to the positioning system.

U.S. Pat. No. 9,510,796 teaches an intraoral x-ray sensor which includes a sensor housing and a universal serial bus (USB) data cable. The sensor housing has an opening. The USB data cable includes an outer sheath and a first data line, a second data line, a ground line, a power line, and at least two independent fillers positioned within the outer sheath. In one embodiment, at least two lines selected from the group including the first data line, the second data line, the ground line, and the power line are twisted together to form a single bundle. The opening receives the data cable.

Referring to FIG. 30 U.S. Pat. No. 9,510,796 also teaches a dental x-ray system 610 includes an x-ray source 612 which is located on an end 613 of a mechanical arm 615. When activated, the x-ray source 612 generates an x-ray stream 616 that has a generally circular cross-section. In many applications, a collimator is used to reduce the size of the stream and generate a smaller x-ray stream having a rectangular cross-section. A collimator may be used with a mechanical positioning device to help align the x-ray stream with an x-ray sensor. The x-ray source 612 is positioned by an operator so that the x-ray stream 616 is directed to an intraoral sensor 620. The intraoral sensor 620 is shown located in the mouth of a patient 621.

The intraoral sensor 620 includes a scintillator that coverts x-ray radiation to visible light. The intraoral sensor 620 may also be configured to convert x-rays into electric charge without a scintillator. Unless otherwise specified, the term pixel refers both to a pixel in the array of pixels that converts x-rays to electrons without a scintillator and a pixel in the array of pixels and its associated scintillator or portion of a scintillator.

Still referring to FIG. 30 a wire, cable, or similar connector 627 connects the intraoral sensor 620 to a computer 630. The computer 630 includes various components, including a processor or similar electronic device 632, an input/output interface 634, and memory 636, RAM and ROM. The input/output interface 634 is a USB connection and the cable 627 is a USB cable. Image data captured by the intraoral sensor 620 and processed by the computer 630 is sent to a display 638 and viewed as image 640.

The inventor hereby incorporates by reference the above-referenced patents.

### SUMMARY OF THE INVENTION

The present invention is a sensor positioner for use with a dental digital radiography system which includes an x-ray source which transmits through an x-ray cone an x-ray stream which has a generally circular cross-section and an intraoral sensor which is aligned with the x-ray source. The sensor positioner includes an aiming ring and a sensor carrier. The aiming ring includes a receptacle which is a single hole having a predefined cross-sectional shape with two axes of symmetry wherein the aiming ring is capable to being disposed adjacent to and visually aligned with to the x-ray source through the x-ray cone. The sensor carrier includes an attachment rod which has a first end and a second end and which has the same shape as the receptacle of the aiming ring so that the receptacle receives the attachment rod at the first end in one of two ways. The sensor holder is coupled to the second end of the attachment rod to integrally form the sensor carrier. The sensor holder has a surface on which the intraoral sensor is placed which visually aligns the intraoral sensor and a clip which secures the dental sensor to the surface so that the intraoral sensor does not slide relative to the center of the aiming ring.

The first aspect of the present invention is to provide a dental sensor positioning system for dental x-ray procedures which secures a dental sensor in a defined and accurate position thereby facilitating accurate x-rays.

The second aspect of the present invention is to provide a dental sensor positioning system which is easier to use than current available dental sensor positioning systems.

The third aspect of the present invention is to provide a dental sensor positioning system which does not include numerous moving parts thereby resulting in stable dental x-ray procedures.

The fourth aspect of the present invention is to provide a dental sensor positioning system which is stable thereby eliminating unnecessary radiation exposure and patient discomfort.

The fifth aspect of the present invention is to provide a dental sensor positioning system which eliminates movement between the x-ray source and the dental sensor located within the patient's mouth thereby producing accurate results.

The sixth aspect of the present invention is to provide a dental sensor positioning system which facilitates exposure of all four corners of the dental sensor.

The seventh aspect of the present invention is to provide a dental sensor positioning system which includes a comfortable cushion cover that may be reused for the entire series of x-rays for the same patient.

The eighth aspect of the present invention is to provide a dental sensor positioning system which facilitates the use of disposable bite blocks thwarting the spread of infection and communicable diseases.

The ninth aspect of the present invention is to provide a dental sensor positioning system which is generally more comfortable to the patient.

The tenth aspect of the present invention is to provide a dental sensor positioning system which is cost efficient to manufacture, in which the bite blocks are autoclavable and which and includes disposable covers.

The eleventh aspect of the present invention is to provide a dental sensor positioning system which eliminates the complicated film carrier selection and mounting process associated with prior art dental sensor positioning systems.

The twelfth aspect of the present invention is to provide a dental sensor positioning system which eliminates the uncertainty associated with correctly aligning the many different types of sensor holders in a patient's mouth with respect to the x-ray tube.

The thirteenth aspect of the present invention is to provide a dental sensor positioning system which is fool-proof in that the sensor holders can only be inserted into the aiming device only one of two ways.

The fourteenth aspect of the present invention is to provide a dental sensor positioning system for image recording in the oral cavity which, with respect to often hectic procedures in a dental practice or dental clinic, is especially user-friendly.

Other aspects and many of the attendant advantages will be more readily appreciated as the same becomes better understood by reference to the following detailed description and considered in conjunction with the accompanying drawing in which like reference symbols designate like parts throughout the figures.

### DESCRIPTION OF THE DRAWINGS

FIG. 1 is a schematic diagram of a film positioning system in accordance with U.S. Pat. No. 5,327,477 which includes an aiming ring and a film carrier.

FIG. 2 is a schematic diagram of an annotated film carrier in accordance with U.S. Pat. No. 5,327,477.

FIG. 3 is a perspective view of the annotated film carrier of FIG. 2 which is positioned in the aiming ring of FIG. 1.

FIG. 4 is a more detailed side elevation of a film carrier in accordance with U.S. Pat. No. 5,327,477 showing frictional support means for holding the x-ray film about the support plate.

FIG. 5 is a perspective view of a sleeve placed about the film holder in accordance with U.S. Pat. No. 5,327,477 for increased patient comfort.

FIG. 6 is a rear view of an aiming ring in accordance with U.S. Pat. No. 5,327,477.

FIG. 7 is a partial side view of a dental x-ray tube on which the aiming ring of FIG. 6 is placed during the x-ray procedure.

FIG. 8 is a side view of an anterior film carrier in accordance with U.S. Pat. No. 5,327,477.

FIG. 9 is a schematic diagram of a posterior upper left, lower right film carrier in accordance with U.S. Pat. No. 5,327,477.

FIG. 10 is a schematic diagram of an upper right, lower left film carrier in accordance with U.S. Pat. No. 5,327,477.

FIG. 11 is a side view of a bite-wing film carrier in accordance with U.S. Pat. No. 5,327,477.

FIG. 12 is a perspective view of the film positioning system in accordance with U.S. Pat. No. 5,327,477 deployed on an x-ray cone in the proper orientation for filming the upper anterior teeth;

FIG. 13 is a perspective view of the film positioning system in accordance with U.S. Pat. No. 5,327,477 deployed on an x-ray cone in another position.

FIG. 14 is a flow diagram of the film positioning method in accordance with U.S. Pat. No. 5,327,477.

FIG. 15 illustrates an isometric assembly view of a dental positioning system in accordance with U.S. Pat. No. 9,314,215 as viewed from a perspective looking towards a side, rear and top thereof.

FIG. 16 illustrates the dental positioning system of FIG. 15 as viewed from another perspective.

FIG. 17 illustrates a perspective view of the dental positioning system of FIG. 15 in an assembled condition, and in which the dental positioning system is arranged for anterior x-ray images.

FIG. 18 is an elevation view of an alignment ring of the dental positioning system of FIG. 15 viewed from a perspective looking towards a rear side thereof.

FIG. 19 is a perspective view of a film positioning system for dental x-ray procedures in accordance with U.S. Pat. No. 5,652,779 which includes an aiming device.

FIG. 20 is a perspective view showing the orientation of the aiming device of FIG. 19 when an upper left/lower right or upper right/lower left film holder is positioned therein.

FIG. 21 is a perspective view of the film positioning system of FIG. 19 showing the orientation of the aiming device when an anterior film holder is positioned in the aiming device.

FIG. 22 is a perspective view of the film positioning system of FIG. 19 when a bite wing film holder is positioned in the aiming device; FIG. 23 is a perspective view of the film positioning system of FIG. 19 shown useful for digital radiography.

FIG. 24 is a side elevation of a sensor for image recording in the oral cavity having a signal line extending from the back side;

FIG. 25 is a side elevation of a sensor holder in accordance with U.S. Pat. No. 5,677,537 with a bite section for receiving the sensor of FIG. 24.

FIG. 26 a partial cross-sectional view of the holder of FIG. 25 taken along the line III-III of FIG. 25.

FIG. 27 a partial cross-sectional view of a holder similar to the holder of FIG. 27 for a landscape imaging format; FIG. 28 is a side elevation of a device including the sensor of FIG. 24 and the holder of FIG. 25 including a hose slipped over the tube parts for fixing the parts relative to one another.

FIG. 29 is a side elevation of another holder in accordance with U.S. Pat. No. 5,677,537 with a bite section for receiving the sensor of FIG. 24 in a representation corresponding to the view of FIG. 25.

FIG. 30 is a schematic illustration of a dental x-ray system including an x-ray source, an intraoral sensor located in a patient's mouth and a computer connected to the intraoral sensor. in accordance with U.S. Pat. No. 9,510,796.

FIG. 31 is a perspective drawing of a first sensor positioner including an aiming ring having a receptacle and a first carrier having a first sensor holder according to the first embodiment of the present invention.

FIG. 32 is a schematic drawing of the first sensor positioner of FIG. 31 a diagram showing placement of the first sensor holder for a maxillary anterior positioning on the patient's lower teeth.

FIG. 33 is a schematic drawing of the first sensor positioner of FIG. 31 and a diagram showing placement of the first sensor holder for a maxillary anterior positioning on the patient's upper teeth.

FIG. 34 is a perspective drawing of a second sensor positioner including an aiming ring having a receptacle and a second carrier having a second sensor holder according to the second embodiment of the present invention.

FIG. 35 is a schematic drawing of the second sensor positioner of FIG. 34 and a diagram showing placement of the second sensor holder for a mandibular posterior positioning on one side of the patient's lower teeth.

FIG. 36 is a schematic drawing of the first sensor positioner of FIG. 34 and a diagram showing placement of the second sensor holder for a mandibular posterior positioning on the other side of the patient's lower teeth.

FIG. 37 is a perspective drawing of a third sensor positioner including an aiming ring having a receptacle and a third carrier having a third sensor holder according to the third embodiment of the present invention.

FIG. 38 is a schematic drawing of the third sensor positioner of FIG. 37 and a diagram showing placement of the third sensor holder for a maxillary posterior positioning on one side of the patient's upper teeth.

FIG. 39 is a schematic drawing of the third sensor positioner of FIG. 37 and a diagram showing placement of the third sensor holder for a maxillary

posterior positioning on the other side of the patient's upper teeth.

FIG. 40 is a perspective drawing of a fourth sensor positioner including an aiming ring having a receptacle and a fourth carrier having a fourth sensor holder according to the fourth embodiment of the present invention.

FIG. 41 is a schematic drawing of the fourth sensor positioner of FIG. 40 and a diagram showing placement of the fourth sensor holder for a vertical bitewing positioning on one side of the patient's mouth.

FIG. 42 is a schematic drawing of the fourth sensor positioner of FIG. 40 and a diagram showing placement of the fourth sensor holder for a vertical bitewing positioning on the other side of the patient's mouth.

FIG. 43 is a photograph of the fourth sensor positioner of FIG. 40 showing a clip which secures the dental sensor to the surface so that the intraoral sensor does not slide relative to the center of the aiming ring.

FIG. 44 is a perspective drawing of a fifth sensor positioner including an aiming ring having a receptacle and a fifth carrier having a fifth sensor holder according to the fifth embodiment of the present invention.

FIG. 45 is a schematic drawing of the fifth sensor positioner of FIG. 44 and a diagram showing placement of the fifth sensor holder for a horizontal bitewing positioning on one side of the patient's mouth.

FIG. 46 is a schematic drawing of the fifth sensor positioner of FIG. 44 and a diagram showing placement of the fifth sensor holder for a horizontal bitewing positioning on the other side of the patient's mouth.

FIG. 47 is a schematic drawing of the first through fifth sensor positioners of FIG. 31, FIG. 34, FIG. 37, FIG. 40 and FIG. 44 including the aiming ring and the first carrier through fifth carrier having the first sensor holder through the fifth sensor holder, respectively.

FIG. 48 is a perspective drawing of a sixth sensor positioner including an aiming ring having a receptacle and a sixth carrier having a sixth sensor holder according to the sixth embodiment of the present invention.

FIG. 49 is a perspective drawing of a seventh sensor positioner including an aiming ring having a receptacle and a seventh carrier having a seventh sensor holder according to the seventh embodiment of the present invention.

FIG. 50 is a perspective drawing of an eighth sensor positioner including an aiming ring having a receptacle and an eighth carrier having an eighth sensor holder according to the eighth embodiment of the present invention.

FIG. 51 is a perspective drawing of a ninth sensor positioner including an aiming ring having a receptacle and a ninth carrier having a ninth sensor holder according to the ninth embodiment of the present invention.

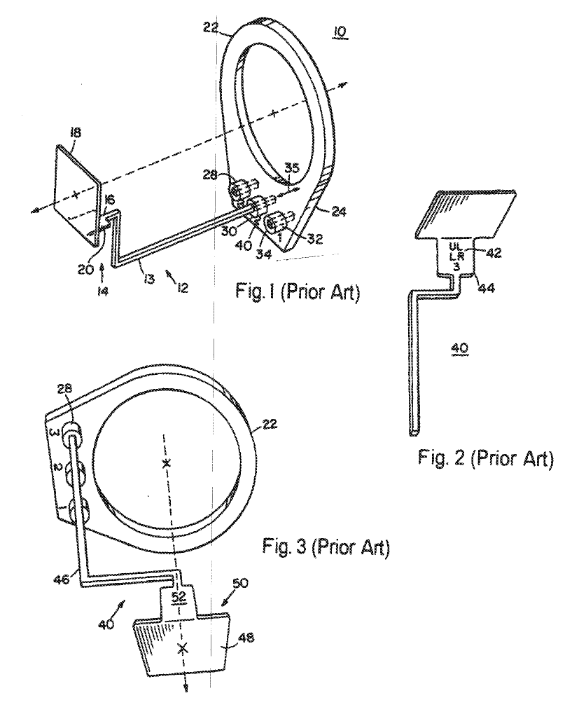

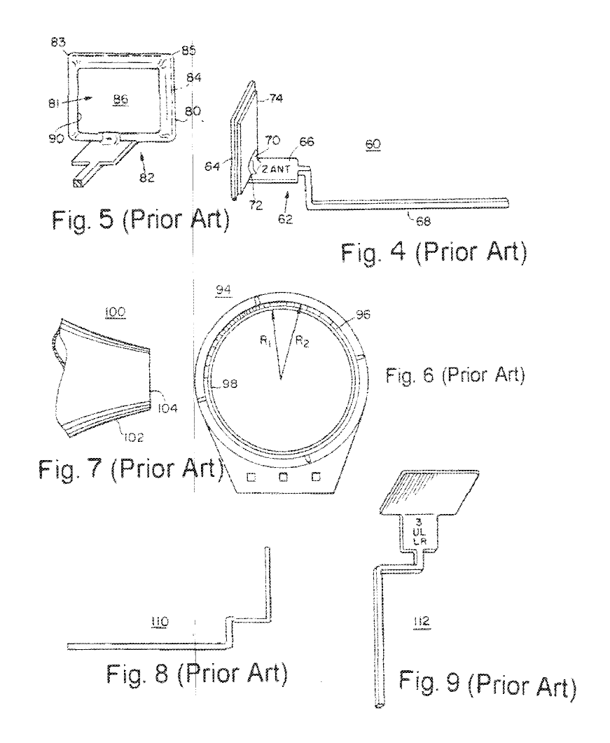

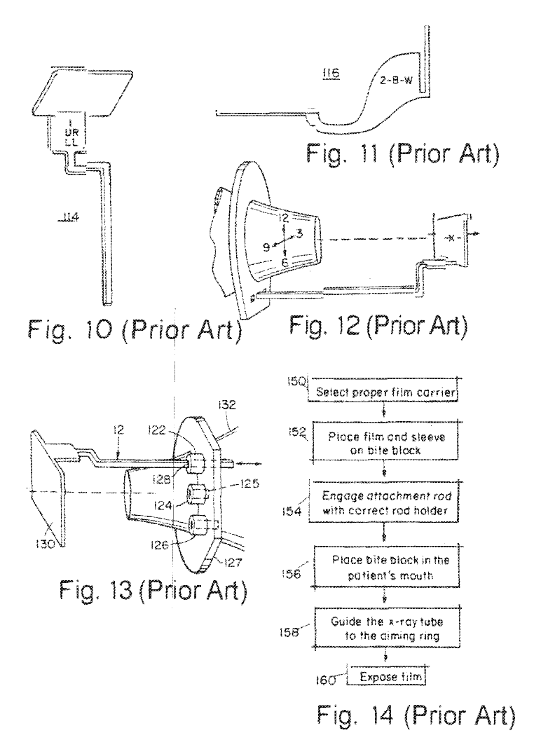

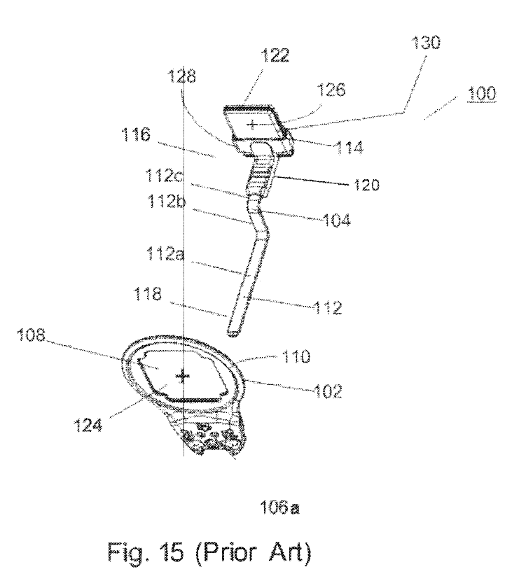

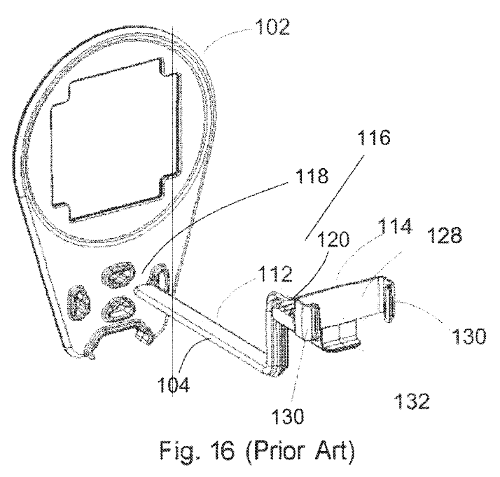

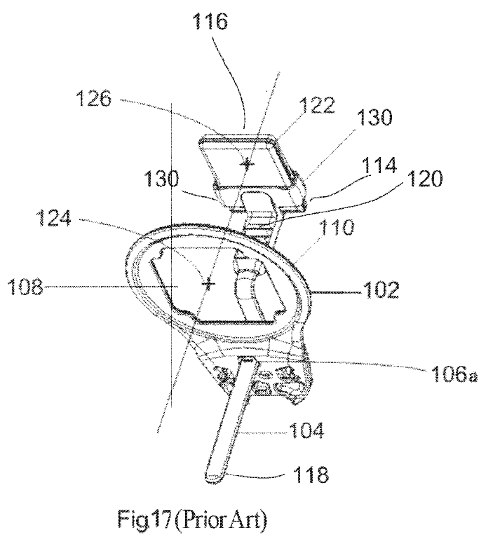

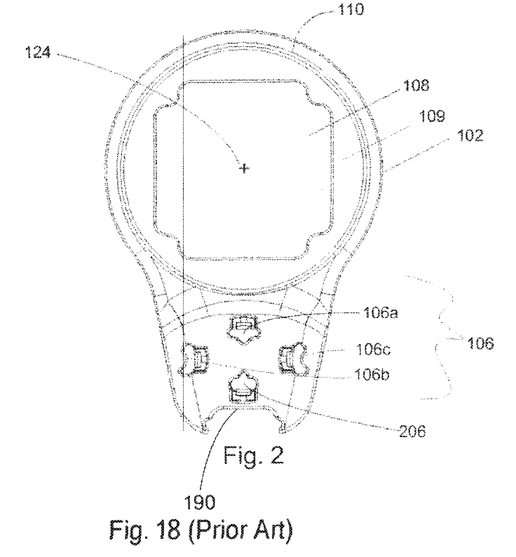

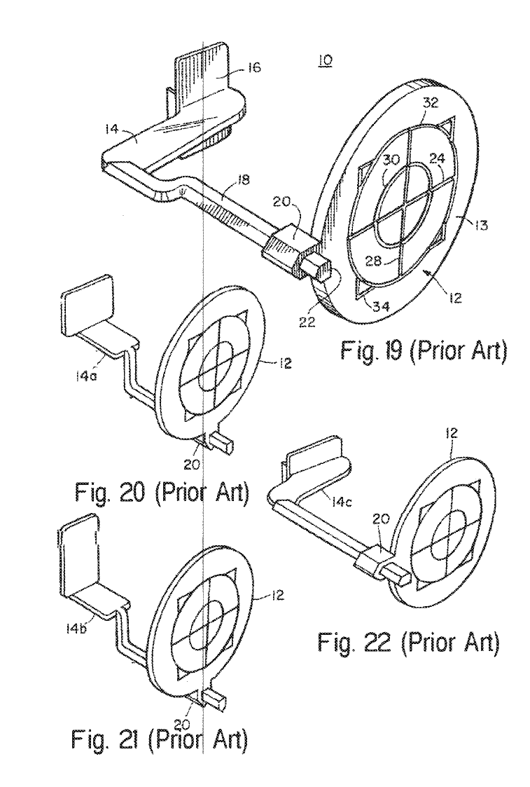

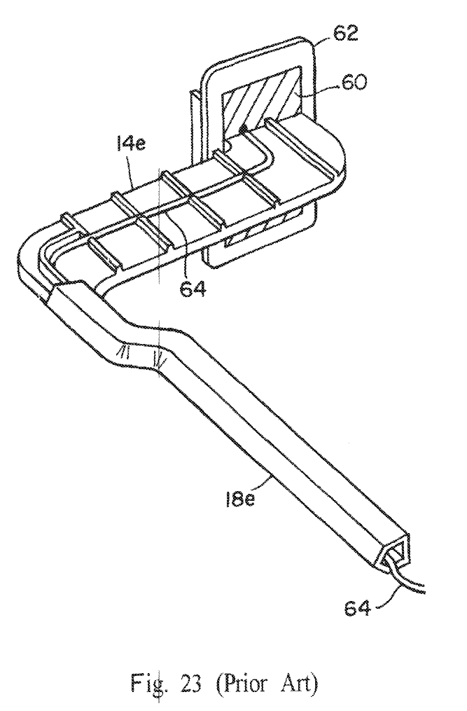

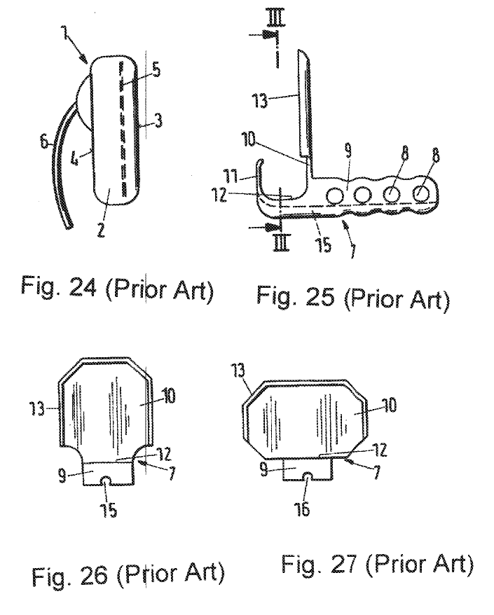

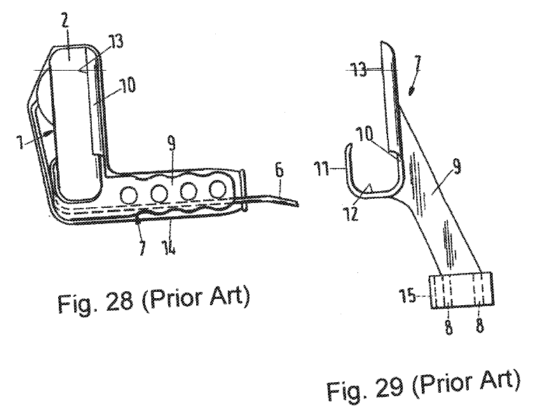

### DESCRIPTION OF THE PREFERRED EMBODIMENTS

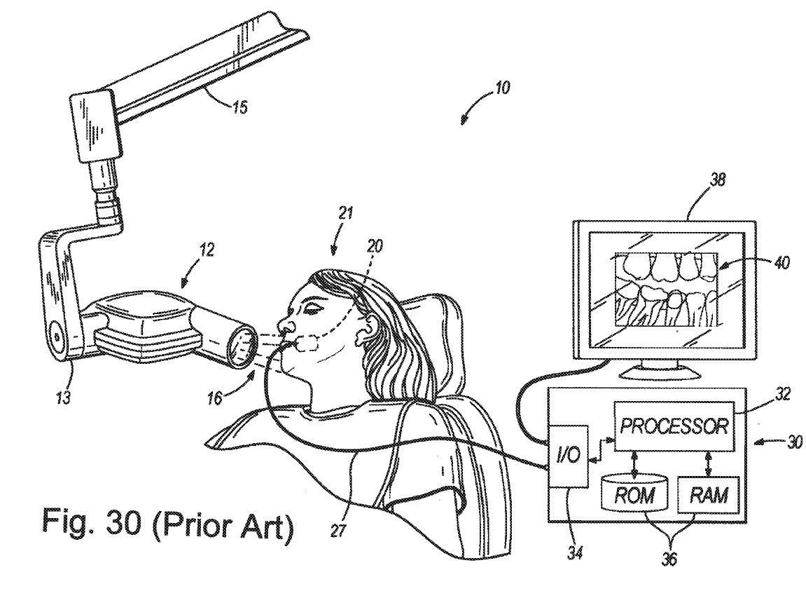

Referring again to FIG. 30 a dental x-ray system 610 includes an x-ray source and an intraoral sensor 620. The x-ray source transmits through an x-ray cone an x-ray stream which has a generally circular cross-section. The intraoral sensor 620 is a digital sensor and is coupled to the x-ray cone of the x-ray source.

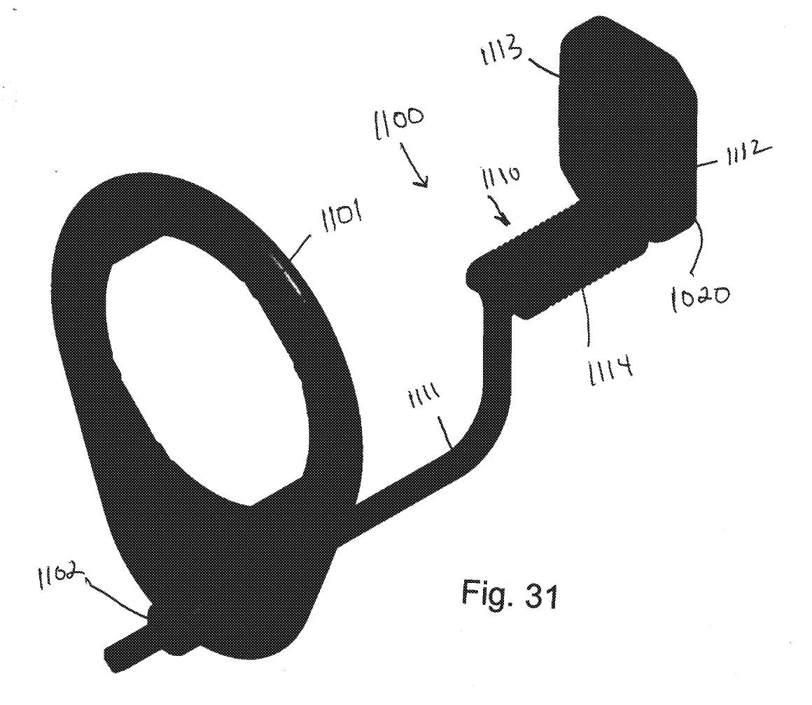

Referring to FIG. 31 in conjunction with FIG. 30 a first sensor positioner 1100 includes an aiming ring 1101 having a receptacle 1102. The receptacle 1102 has a predefined cross-sectional shape with, at most, only one axis of symmetry. The aiming ring 1101 is capable to being attached to the x-ray cone of the dental x-ray system 610 The first sensor positioner 1100 also includes a first carrier 1110. The first carrier 1110 includes a first attachment rod 1111 and a first sensor holder 1112. The first attachment rod 1111 extends from the first sensor holder 1112. The first attachment rod 1111 has the same shape as the receptacle 1102 such that the first attachment rod 1111 is received in the receptacle 1102 only one way. The first sensor holder 1112 has a surface 1113 that securely aligns the intraoral sensor 620 to which it is attached so that the intraoral sensor 620 does not slide relative to the center of the aiming ring 1101. The first sensor holder 1112 has a bite-block 1114. The first sensor holder 1112 is an anterior holder which is configured to take images to teeth in the anterior portion of a patient's oral cavity. The first carrier 1110 may be color-coded blue.

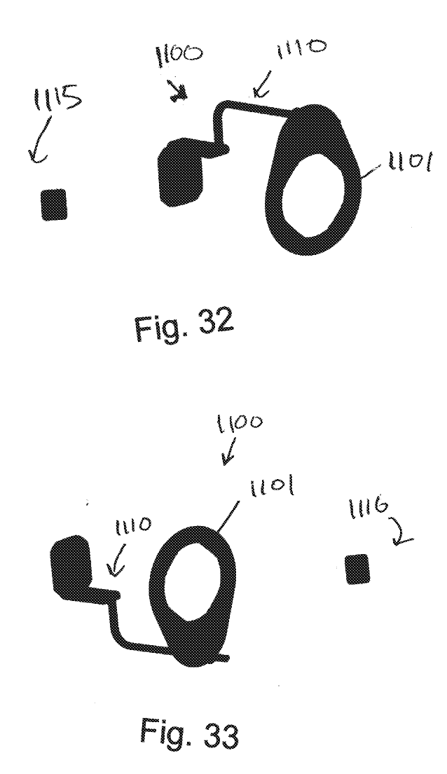

Referring to FIG. 32 in conjunction with FIG. 31 the user refers to a diagram 1115 for a maxillary anterior positioning on the patient's lower teeth. The user positions the first sensor holder 1112 so that it is parallel to the roots of the teeth and places it either under the tongue or over the tongue, whichever is most comfortable for the patient. If large mandibular tori are present, the user positions the first sensor holder 1112 behind the tori, even if it is in the first molar area asking the patient to close end-to-end to maintain the placement. If the first sensor holder 1112 does not remain parallel to the roots of the incisors, the user bisects his angle. Should the incisal edges be missing when acquiring his images, the user places a cotton roll or flap between the tooth and the first sensor holder 1112 and/or verifies that the first sensor holder 1112 is parallel to the roots.

Referring to FIG. 33 in conjunction with FIG. 31 the user refers to a diagram 1116 for a maxillary anterior positioning on the patient's upper teeth. The user positions the sensor holder 1112 so that it is parallel to the roots of the teeth and places it either under the tongue or over the tongue, whichever is most comfortable for the patient. If large mandibular tori are present, the user positions the first sensor holder 1112 behind the tori, even if it is in the first molar area asking the patient to close end-to-end to maintain the placement. If the first sensor holder 1112 does not remain parallel to the roots of the incisors, the user bisects his angle. Should the incisal edges be missing when acquiring his images, the user places a cotton roll or flap between the tooth and the first sensor holder 1112 and/or verifies that the first sensor holder 1112 is parallel to the roots.

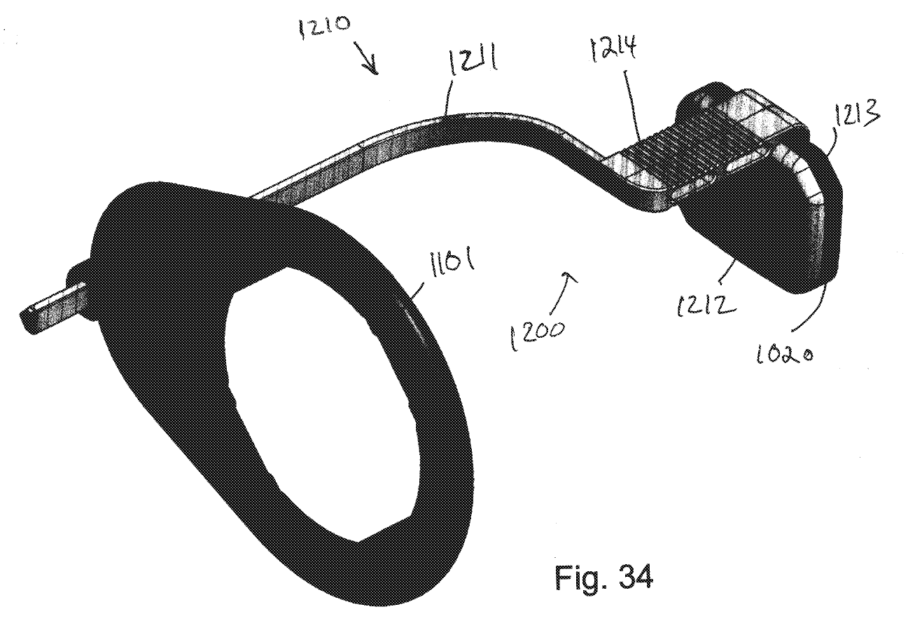

Referring to FIG. 34 in conjunction with FIG. 30 a second sensor positioner 1200 includes the alignment ring 1101 and a second carrier 1210. The second carrier 1210 includes a second attachment rod 1211 and a second first sensor holder 1212. The second attachment rod 1211 extends from the second sensor holder 1212. The second attachment rod 1211 has the same shape as the receptacle 1102 such that the second attachment rod 1211 is received in the receptacle 1102 only one way. The second sensor holder 1212 has a surface 1213 that securely aligns the intraoral sensor 620 to which it is attached so that the intraoral sensor 620 does not slide relative to the center of the aiming ring 1101. The second sensor holder 1212 has a bite-block 1214. The second sensor holder 1212 is an anterior holder which is configured to take images to teeth in the mandibular posterior portion of a patient's lower teeth. The second carrier 1210 may be color-coded yellow.

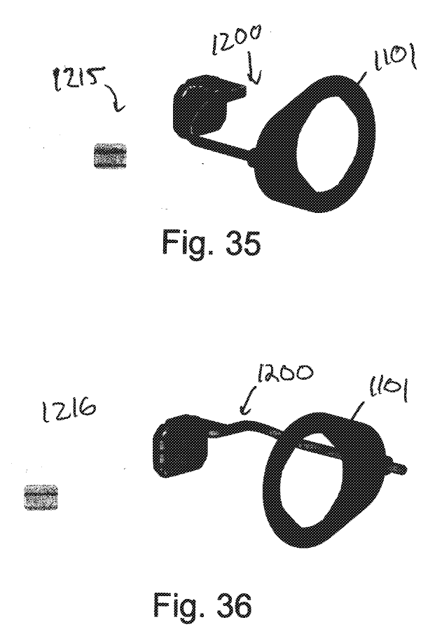

Referring to FIG. 35 in conjunction with FIG. 34 the user refers to a diagram 1215 for a mandibular posterior positioning on one side of the patient's lower teeth. The user positions the second sensor holder 1212 so that it is parallel to the roots of the teeth and places it either under or over the tongue, whichever is most comfortable for the patient. Once he has established that the second sensor holder 1212 is parallel to the roots and parallel to the occlusal line, he moves the second sensor holder 1212 to the mid-line while asking the patient to close. In an event that the second sensor holder 1212 does not remain parallel to the roots of the premolars, he bisects his angle.

Referring to FIG. 36 in conjunction with FIG. 34 the user refers to a diagram 1216 for a mandibular posterior positioning on the other side of the patient's lower teeth. The user positions the second sensor holder 1212 so that it is parallel to the roots of the teeth and places it either under or over the tongue, whichever is most comfortable for the patient. Once he has established that the sensor holder 1212 is parallel to the roots and parallel to the occlusal line, he moves the second sensor holder 1212 to the mid-line while asking the patient to close. In an event that the second sensor holder 1212 does not remain parallel to the roots of the premolars, he bisects his angle.

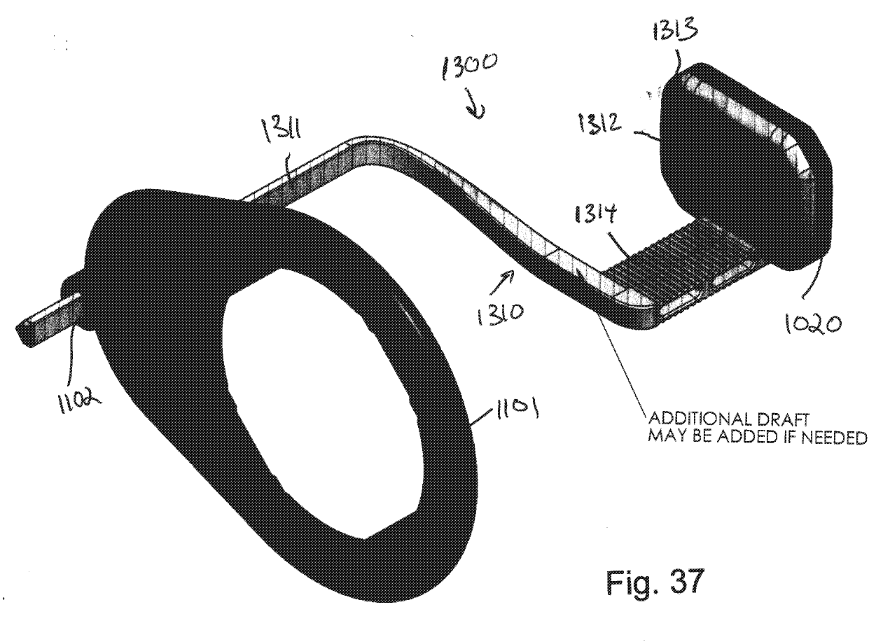

Referring to FIG. 37 in conjunction with FIG. 30 a third sensor positioner 1300 includes the alignment ring 1101 and a third carrier 1310. The third carrier 1310 includes a third attachment rod 1311 and a third sensor holder 1312. The third attachment rod 1311 extends from the third sensor holder 1312. The third attachment rod 1311 has the same shape as the receptacle 1102 such that the third attachment rod 1311 is received in the receptacle 1102 only one way. The third sensor holder 1312 has a surface 1313 that securely aligns the intraoral sensor 620 to which it is attached so that the intraoral sensor 620 does not slide relative to the center of the aiming ring 1101. The third sensor holder 1312 has a bite-block 1314. The third sensor holder 1312 is an anterior holder which is configured to take images to teeth in the mandibular posterior portion of a patient's upper teeth. The second carrier 1210 may be color-coded yellow.

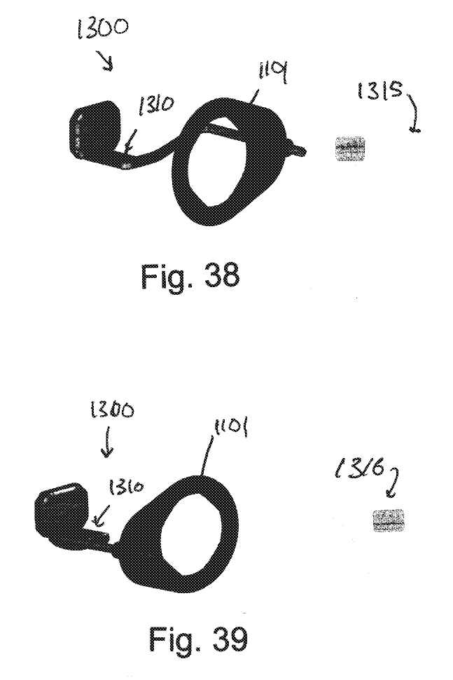

Referring to FIG. 38 in conjunction with FIG. 37 the user refers to a diagram 1315 for a maxillary posterior positioning on one side of the patient's upper teeth. The user positions the third sensor holder 1312 so that it is parallel to the roots of the teeth. Once he has established that the third sensor holder 1312 is parallel to the roots and parallel to the occlusal line, he moves the sensor holder 1312 toward the center of the palate asking the patient to close. If the third sensor holder 1312 does not remain parallel to the roots, he bisects his angle. If a large maxillary torus is present and he is unable to place the third sensor holder 1312 in a traditional position, he places the third sensor holder 1312 on the torus as he would place traditional film and he must bisect his angle.

Referring to FIG. 39 in conjunction with FIG. 37 the user refers to a diagram 1316 for a maxillary posterior positioning on the other side of the patient's upper teeth. The user positions the third sensor holder 1312 so that it is parallel to the roots of the teeth. Once he has established that the third sensor holder 1312 is parallel to the roots and parallel to the occlusal line, he moves the third sensor holder 1312 toward the center of the palate asking the patient to close. If the third sensor holder 1312 does not remain parallel to the roots, he bisects his angle. If a large maxillary torus is present and he is unable to place the third sensor holder 1312 in a traditional position, he places the third sensor holder 1312 on the torus as he would place traditional film and he must bisect his angle.

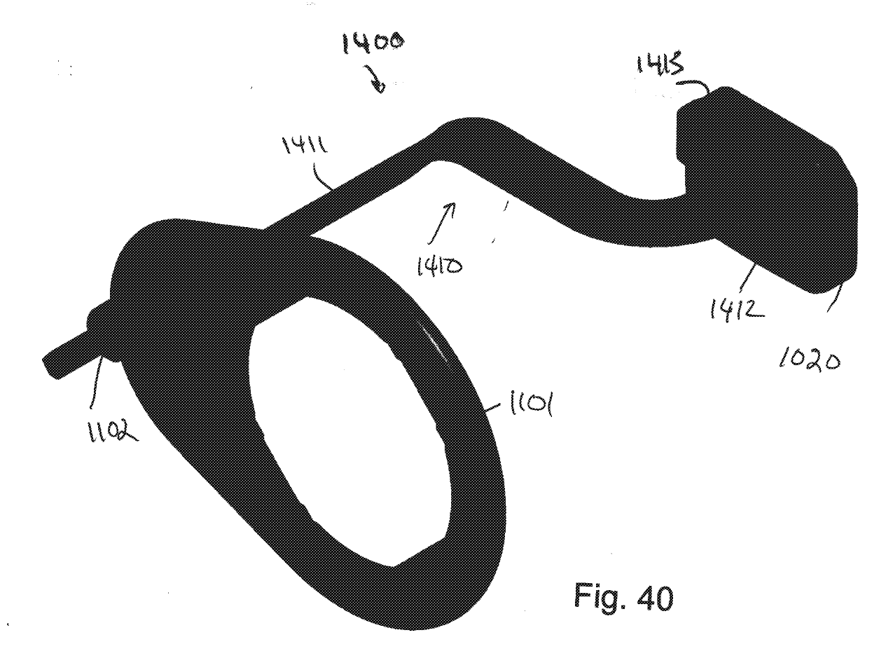

Referring to FIG. 40 in conjunction with FIG. 30 a fourth sensor positioner 1400 includes the alignment ring 1101 and a fourth carrier 1410. The fourth carrier 1410 includes a fourth attachment rod 1411 and a fourth sensor holder 1412. The fourth attachment rod 1411 extends from the fourth sensor holder 1412. The fourth attachment rod 1411 has the same shape as the receptacle 1102 such that the fourth attachment rod 1411 is received in the receptacle 1102 only one way. The fourth sensor holder 1412 has a surface 1413 that securely aligns the intraoral sensor 620 to which it is attached so that the intraoral sensor 620 does not slide relative to the center of the aiming ring 1101. The fourth sensor holder 1412 is a horizontal bitewing holder which is configured to take horizontal bitewing images. The fourth carrier 1410 may be color-coded red.

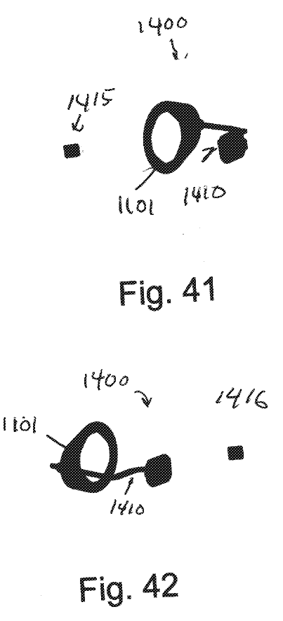

Referring to FIG. 41 in conjunction with FIG. 40 the user refers to a diagram 1415 for a horizontal bitewing positioning of the patient's upper and lower teeth on one side of his mouth. The user positions the fourth sensor holder 1412 so that it is parallel to the occlusal line and moves the sensor holder 1412 toward the midline asking the patient to close.

Referring to FIG. 42 in conjunction with FIG. 40 the user refers to a diagram 1416 for a horizontal bitewing positioning of the patient's upper and lower teeth on the other side of his mouth. The user positions the fourth sensor holder 1412 so that it is parallel to the occlusal line and moves the sensor holder 1412 toward the midline asking the patient to close.

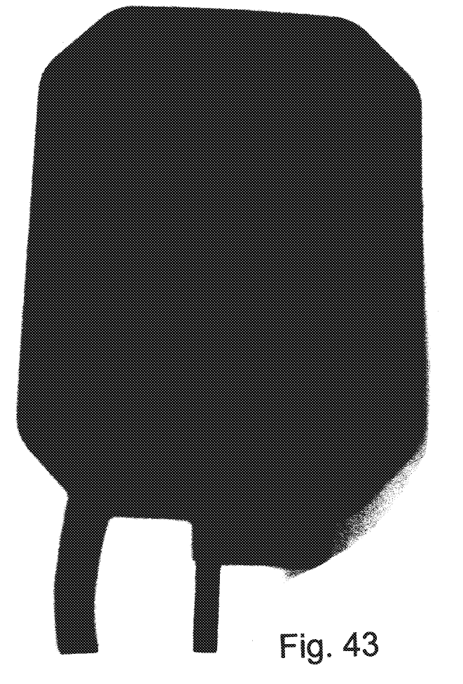

Referring to FIG. 43 in conjunction with FIG. 40 the fourth sensor holder 1412 also includes a clip 1420 for securing the intraoral sensor 620 to the surface 1413 of the fourth sensor holder 1412 so that the intraoral sensor 620 does not slide relative to the center of the alignment ring 1101.

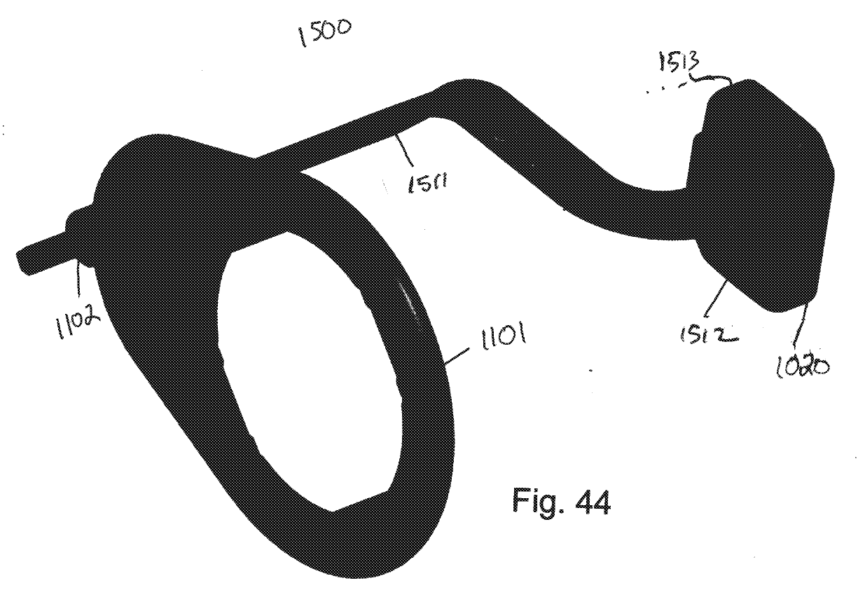

Referring to FIG. 44 in conjunction with FIG. 30 a fifth sensor positioner 1500 includes the alignment ring 1101 and a fifth carrier 1510. The fifth carrier 1510 includes a fifth attachment rod 1511 and a fifth sensor holder 1512. The fifth attachment rod 1511 extends from the fifth sensor holder 1512. The fifth attachment rod 1511 has the same shape as the receptacle 1102 such that the fifth attachment rod 1511 is received in the receptacle 1102 only one way. The fifth sensor holder 1512 has a surface 1513 that securely aligns the intraoral sensor 620 to which it is attached so that the intraoral sensor 620 does not slide relative to the center of the aiming ring 1101. The fifth sensor holder 1512 is a vertical bitewing holder which is configured to take horizontal bitewing images. The fifth carrier 1510 may be color-coded red.

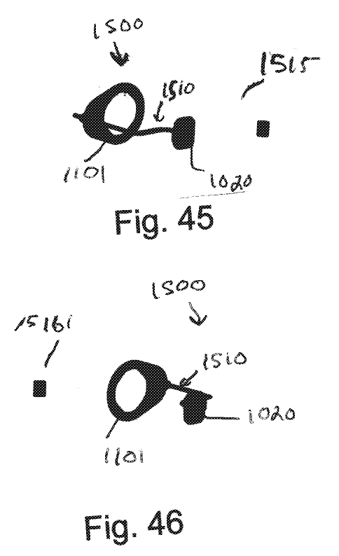

Referring to FIG. 45 in conjunction with FIG. 44 the user refers to a diagram 1515 for a vertical bitewing positioning of the patient's upper and lower teeth on one side of his mouth. The user positions the fifth sensor holder 1512 so that it is parallel to the occlusal line and moves the sensor holder 1512 toward the midline asking the patient to close.

Referring to FIG. 46 in conjunction with FIG. 44 the user refers to a diagram 1516 for a vertical bitewing positioning of the patient's upper and lower teeth on the other side of his mouth. The user positions the fifth sensor holder 1512 so that it is parallel to the occlusal line and moves the sensor holder 1512 toward the midline asking the patient to close.

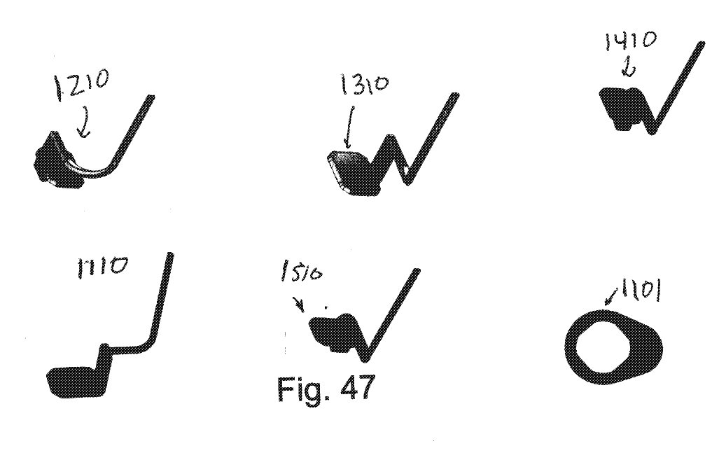

Referring to FIG. 47 in conjunction with FIG. 31, FIG. 34, FIG. 37, FIG. 40 and FIG. 44 a system positioner kit includes the aiming ring 1101, the first sensor positioner 1100, the second sensor positioner 1200, the third sensor positioner 1300, the fourth sensor positioner 1400 and the fifth sensor positioner 1100. Each of the first sensor positioner through the fifth sensor positioner 1100, 1200, 1300, 1400 and 1500 is associated with one of five sets of two diagrams 1115 and 1116, 1215 and 1216, 1315 and 1316, 1415 and 1416, and 1515 and 1516, respectively. A dental technician uses these diagrams to not only select the correct sensor positioner, but to also assist him properly setting up the selected sensor positioner.

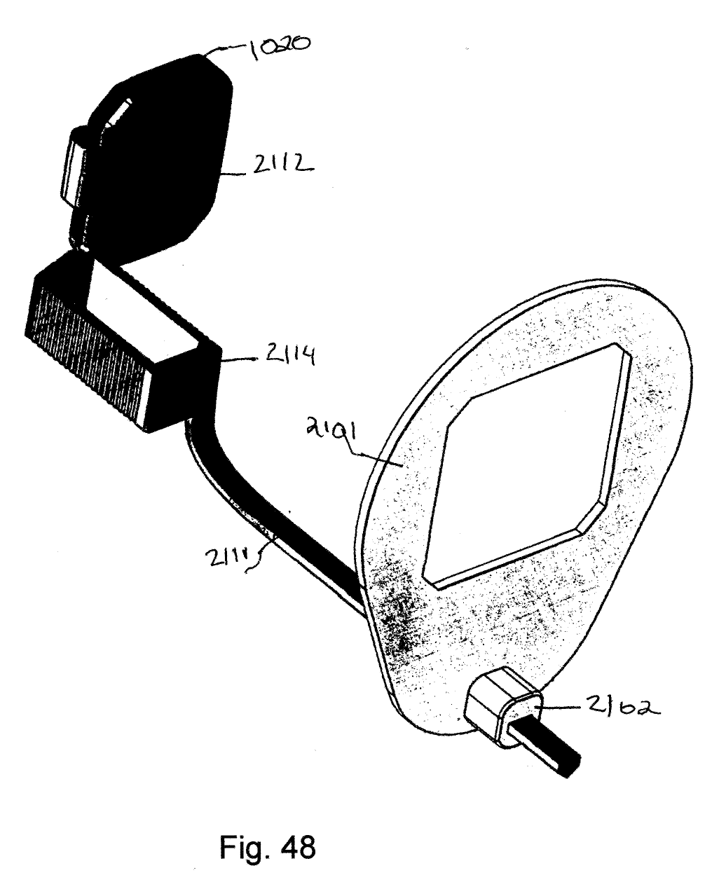

Referring to FIG. 48 in conjunction with FIG. 30 a first horizontal endo sensor positioner 2100 includes an alignment ring 2101 with a receptacle 2102 and a first horizontal endo carrier 2110. The first horizontal endo carrier 2110 includes a first horizontal endo attachment rod 2111 and a first horizontal endo sensor holder 2112. The first horizontal endo attachment rod 2111 extends from the first horizontal endo sensor holder 2112. The first horizontal endo attachment rod 2111 has the same shape as the receptacle 2102 such that the first horizontal endo attachment rod 2111 is received in the receptacle 2102 only one way. The first horizontal endo sensor holder 2112 has a surface 2113 that securely aligns the intraoral sensor 620 to which it is attached so that the intraoral sensor 620 does not slide relative to the center of the aiming ring 2101. The first horizontal endo sensor holder 2112 has a bite-block 2114.

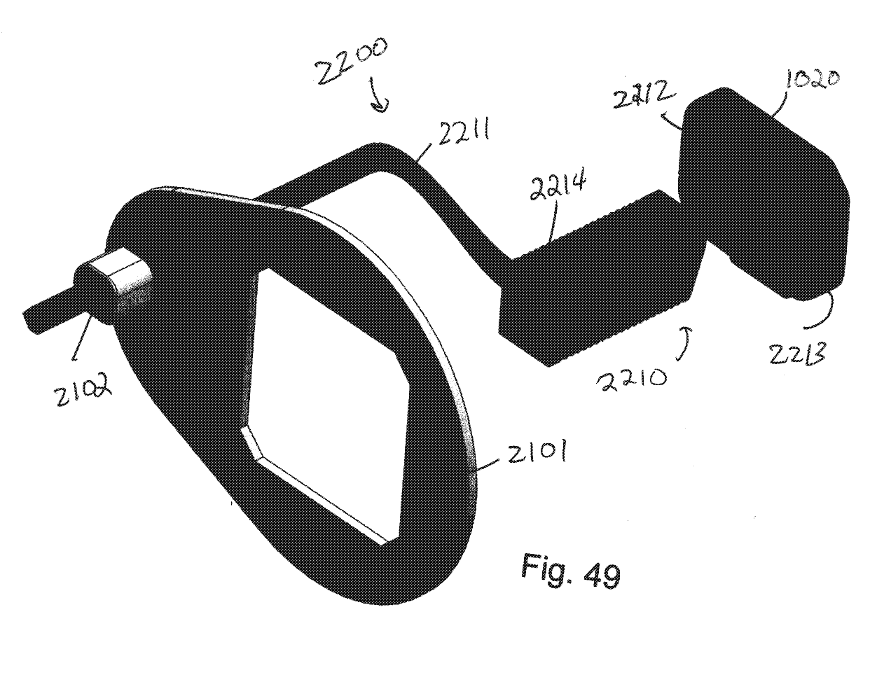

Referring to FIG. 49 in conjunction with FIG. 30 a second horizontal endo sensor positioner 2200 includes an alignment ring 2201 with a receptacle 2202 and a second horizontal endo carrier 2210. The second horizontal endo carrier 2210 includes a second horizontal endo attachment rod 2211 and a second horizontal endo sensor holder 2212. The second horizontal endo attachment rod 2211 extends from the second horizontal endo sensor holder 2212. The second horizontal endo attachment rod 2211 has the same shape as the receptacle 2202 such that the second horizontal endo attachment rod 2211 is received in the receptacle 2202 only one way. The second horizontal endo sensor holder 2212 has a surface 2213 that securely aligns the intraoral sensor 620 to which it is attached so that the intraoral sensor 620 does not slide relative to the center of the aiming ring 2201. The second horizontal endo sensor holder 2212 has a bite-block 2214.

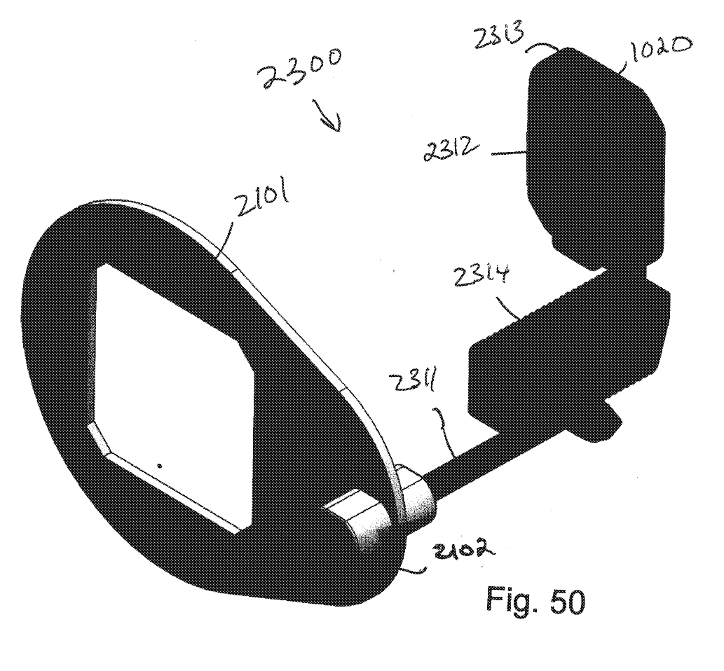

Referring to FIG. 50 in conjunction with FIG. 30 a first vertical endo sensor positioner 2300 includes an alignment ring 2301 with a receptacle 2302 and a first vertical endo carrier 2310. The first vertical endo carrier 2310 includes a first vertical endo attachment rod 2311 and a first vertical endo sensor holder 2312. The first vertical endo attachment rod 2311 extends from the first vertical endo sensor holder 2312. The first vertical endo attachment rod 2311 has the same shape as the receptacle 2302 such that the first vertical endo attachment rod 2311 is received in the receptacle 2302 only one way. The first vertical endo sensor holder 2312 has a surface 2313 that securely aligns the intraoral sensor 620 to which it is attached so that the intraoral sensor 620 does not slide relative to the center of the aiming ring 2301. The first vertical endo sensor holder 2312 has a bite-block 2314.

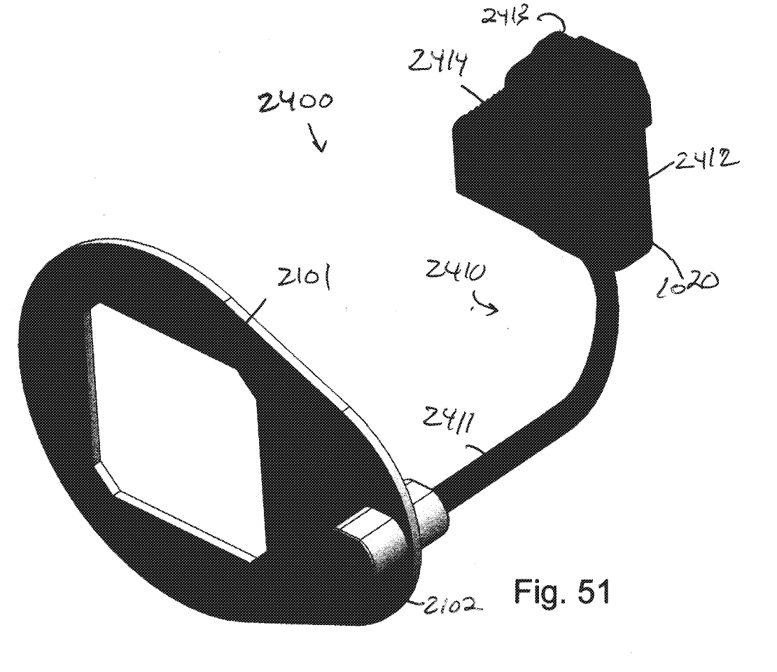

Referring to FIG. 51 in conjunction with FIG. 30 a second vertical endo sensor positioner 2400 includes an alignment ring 2401 with a receptacle 2402 and a second vertical endo carrier 2410. The second vertical endo carrier 2410 includes a second vertical endo attachment rod 2411 and a second vertical endo sensor holder 2412. The second vertical endo attachment rod 2411 extends from the second vertical endo sensor holder 2412. The second vertical endo attachment rod 2411 has the same shape as the receptacle 2402 such that the second vertical endo attachment rod 2411 is received in the receptacle 2402 only one way. The second vertical endo sensor holder 2412 has a surface 2413 that securely aligns the intraoral sensor 620 to which it is attached so that the intraoral sensor 620 does not slide relative to the center of the aiming ring 2401. The second vertical endo sensor holder 2412 has a bite-block 2414.

From the foregoing it can be seen that a sensor positioner including an aiming ring having a receptacle and a carrier having a sensor holder has been described. It should be noted that the sketches are not drawn to scale and that distances of and between the figures are not to be considered significant.

Accordingly it is intended that the foregoing disclosure and showing made in the drawing shall be considered only as an illustration of the principle of the present invention.

What is claimed is:

1. A sensor positioning system for use with a dental digital radiography system including an x-ray source which transmits through an x-ray cone an x-ray stream which has a generally circular cross-section and an intraoral sensor which is aligned with the x-ray source, said sensor positioning system comprising:
a. an aiming ring including a receptacle wherein said receptacle which is a single hole having a predefined cross-sectional shape with two axes of symmetry wherein said aiming ring is capable to being disposed adjacent to and visually aligned with to the x-ray source through the x-ray cone; and
b. a plurality of sensor carriers including:
i. a plurality of attachment rods each of which has a first end and a second end and each of which has the same shape as said receptacle of said aiming ring so that said receptacle receives each of said attachment rods at said first end in one of two ways; and
ii. a plurality of sensor holders each of which is coupled to said second end of one of said attachment rods to integrally form one of said sensor carrier wherein said sensor holder has a surface on which the intraoral sensor is placed which visually aligns the intraoral sensor and a clip which secures the dental sensor to said surface so that the intraoral sensor does not slide relative to the center of said aiming ring.

2. A sensor positioning system according to claim 1 wherein said receptacle of said aiming ring accepts each of said sensor carriers in only two ways so there no confusion into which receptacle to insert each of said sensor carriers.

3. A sensor positioning system according to claim 1 wherein each of carriers includes a set of two diagrams each showing a dental technician placement of said sensor holder with respect to a patient's teeth so that placement is in its proper location.

4. A sensor positioning system according to claim 1 wherein said sensor carrier has an anterior holder which is configured to take images to teeth in the anterior portion of a patient's teeth.

5. A sensor positioning system according to claim 4 wherein said sensor carrier is color-coded and wherein said color-coded carrier is used with said set of two diagrams in combination whereby placement of said sensor holder with respect to a patient's teeth is in its proper location.

6. A sensor positioning system according to claim 3 wherein said sensor carrier has a posterior holder. which is configured to take images to teeth in the posterior portion of a patient's teeth.

7. A sensor positioning system according to claim 6 wherein said sensor carrier is color-coded wherein said color-coded carrier is used with said set of two diagrams in combination whereby placement of said sensor holder with respect to a patient's teeth is in its proper location.

8. A dental digital radiography system according to claim 1 where said sensor holder of said carrier is a bitewing holder. which is configured to take images to teeth in the bitewing portion of a patient's teeth.

9. A dental digital radiography system according to claim 8 where said carrier is color-coded wherein said color-coded carrier is used with said set of two diagrams in combination whereby placement of said sensor holder with respect to a patient's teeth is in its proper location.

10. A sensor positioner for use with a dental digital radiography system including an x-ray source which transmits through an x-ray cone an x-ray stream which has a generally circular cross-section and an intraoral sensor which is aligned with the x-ray source, said sensor positioner comprising:
a. an aiming ring including a receptacle wherein said receptacle which is a single hole having a predefined cross-sectional shape with two axes of symmetry wherein said aiming ring is capable to being disposed adjacent to and visually aligned with to the x-ray source through the x-ray cone; and
b. a sensor carrier including:
i. an attachment rods which has a first end and a second end and each of which has the same shape as said receptacle of said aiming ring so that said receptacle receives attachment rods at said first end in one of two ways; and
ii. a sensor holder which is coupled to said second end of one of said attachment rods to integrally form one of said sensor carrier wherein said sensor holder has a surface on which the intraoral sensor is placed which visually aligns the intraoral sensor and a clip which secures the dental sensor to said surface so that the intraoral sensor does not slide relative to the center of said aiming ring.

11. A sensor positioner according to claim 8 wherein said receptacle of said aiming ring accepts said sensor carrier in only two ways so there no confusion into which receptacle to insert each of said sensor carriers.

12. A sensor positioner according to claim 8 wherein said carrier includes a set of two diagrams each showing a dental technician placement of said sensor holder with respect to a patient's teeth so that placement is in its proper location.

13. A sensor positioner according to claim 8 wherein said sensor carrier has an anterior holder which is configured to take images to teeth in the anterior portion of a patient's teeth.

14. A sensor positioner according to claim 4 wherein said sensor carrier is color-coded and wherein said color-coded carrier 11 is used with said set of two diagrams in combination whereby placement of said sensor holder with respect to a patient's teeth is in its proper location.

15. A sensor positioner according to claim 9 wherein said sensor carrier has a posterior holder. which is configured to take images to teeth in the posterior portion of a patient's teeth.

16. A sensor positioner according to claim 13 wherein said sensor carrier is color-coded wherein said color-coded carrier is used with said set of two diagrams in combination whereby placement of said sensor holder with respect to a patient's teeth is in its proper location.

17. A sensor positioner according to claim 11 wherein said sensor carrier has an anterior holder has a clip for securing the dental sensor to the surface of said anterior holder so that the intraoral sensor does not slide relative to the center of said aiming ring.
